SEQUENCE LISTING (3 sequences)

SEQ ID NO: 1 - DNA, 89 bp, Artificial Sequence
  Exemplary tag library
agaattcgggccttaattaannnnnnnnnnnnnnnnnnnnnnnnnnnnnnnngggcccgcataagtcttcnnnnnnggatccgagtgat

SEQ ID NO: 2 - DNA, 20 bp, Artificial Sequence
  Primer
agaattcgggccttaattaa

SEQ ID NO: 3 - DNA, 18 bp, Artificial Sequence
  Primer
atcactcggatccnnnnn

(19) United States
(12) Patent Application Publication
(10) Pub. No.: US 2005/0260570 A1
(43) Pub. Date: Nov. 24, 2005

(54) Sequencing by proxy

(76) Inventors: Jen-i Mao, Palo Alto, CA (US); Shujun Luo, Castro Valley, CA (US); Alan Ewing, El Cerrito, CA (US); David H. Lloyd, Belmont, CA (US); Stephen C. MacEvicz, Cupertino, CA (US)

Correspondence Address: PERKINS COIE LLP, P.O. BOX 2168, MENLO PARK, CA, 94026, US

(21) Appl. No.: 09/867,201

(22) Filed: May 29, 2001

(51) Int. Cl. C12Q 1/68

(52) U.S. Cl. 435/6

(57) ABSTRACT

The invention provides methods, kits and materials for determinining simultaneously signature sequences of a population of tagged polynucleotides. Size ladders of polynucleotide fragments are generated from the population of tagged polynucleotides that contain a plurality of size classes. After the size classes are separated, tags of the separated fragment are copied and labeled according to the identity of one or more bases at the ends of the fragments. In a preferred embodiment, the labeled tags are then specifically hybridized to plurality of identical microarrays of tag complements such that the tags from different size classes are hybridized to separate microarrays. Signature sequences are determined by signals generated at hybridization sites having the same address on each of the plurality of microarrays.

### FIELD OF THE INVENTION

The invention relates generally to compositions and methods for analyzing nucleic acids, and more particularly, to hybridization-based methods for characterizing nucleic acid populations.

### BACKGROUND OF THE INVENTION

The availability of convenient and efficient methods for the accurate identification of genetic variation and expression patterns among large sets of genes is crucial for understanding the relationship between an organism's genetic make-up and the state of its health or disease, Collins et al, Science, 282: 682-689 (1998). In regard to expression analysis, several powerful techniques have been developed for such analyses that depend either on specific hybridization of probes to microarrays, e.g. Duggan et al, Nature Genetics, 21: 10-14 (1999); Hacia et al, Nature Genetics, 21: 42-47 (1999), or on the counting of tags or signatures of DNA fragments, e.g. Velculescu et al, Science, 270: 484-487 (1995); Brenner et al, Nature Biotechnology, 18: 630-634 (2000). While the former provides the advantages of scale and the capability of detecting a wide range of gene expression levels, such measurements are subject to variability relating to probe hybridization differences and cross-reactivity, element-to-element differences within microarrays, and microarray-to-microarray differences, Audic and Clayerie, Genomic Res., 7: 986-995 (1997); Wittes et al, J. Natl. Cancer Inst. 91: 400401 (1999). On the other hand, the latter methods, which provide digital representations of abundance, are statistically more robust; they do not require repetition or standardization of counting experiments as counting statistics are well-modeled by the Poisson distribution, and the precision and accuracy of relative abundance measurements may be increased by increasing the size of the sample of tags or signatures counted. Unfortunately, however, this property is difficult to realize routinely because of the cost and complexity of implementing large scale efforts to analyze gene expression based on counting sequence tags.

In regard to assessing genetic variation, the primary technique for discovering and assessing sequence variation among individuals is massive and repetitive conventional sequencing, or so-called re-sequencing, e.g. Nickerson et al, Nature Genetics, 19: 233-240 (1998); Taillon-Miller and Kwok, Genome Res., 9: 499-505 (1999); Cargill et al, Nature Genetics, 22: 231-238 (1999). However, the cost of such projects can be prohibitive if any more than a very small fraction of a genome, such as a few “candidate” genes, is analyzed.

In an attempt to improve the efficiency of large-scale sequencing efforts, Brenner, U.S. Pat. No. 5,763,175, describes methods of using oligonucleotide tags to transfer sequence information from templates to specific sites on an array of tag complements, or anti-tags. The method calls for attaching tags to sequencing templates, generating successively shortened amplification products of the templates with PCR primers that anneal to successively larger portions of the templates, copying and labeling the tags associated with each shortened amplification product, and then specifically hybridizing successively the amplified tags to an array of anti-tags to extract a signature sequence for each of the tagged templates. That is, the labeled tags serve as “proxies” for the templates in the hybridization reactions that provide the read-out of signature sequences. Such use of tags obviates the requirement for preparing and carrying out separate sequencing reactions for each template. The tags also permit mixtures of templates to be processed in one or a few reactions, since sequence information is extracted via the labeling and spatial separation of the tags on a hybridization array. Unfortunately, the processing steps disclosed in Brenner are difficult to carry out because they require either large numbers of different PCR primers and a large number of enzymatic steps and/or they require PCR amplifications with degenerate primers which leads to the spurious amplification of mis-primed sequences. Moreover, the hybridization arrays employed by Brenner are limited to those consisting of immobilized microbeads, which means that a single array must be used for all hybridizations in order to generate signature sequences. As complex mixtures of tags typically require two or more hours hybridization time in order to generate detectable signals, signatures of more than a few tens of nucleotides require several days to accumulate.

In view of the above, it would be highly desirable if a signature sequencing technique were available for measuring gene expression and sequence variation that had the capability of massively parallel analysis of large numbers of templates or nucleic acid fragments, but that was free of the shortcomings of current techniques.

### SUMMARY OF THE INVENTION

Accordingly, objects of our invention include, but are not limited to, providing a method and compositions for analyzing gene expression; providing a method of providing a digital representation of relative abundances of polynucleotides in a complex population; providing a method for profiling gene expression of large numbers of genes simultaneously or identifying large numbers of polymorphic genes simultaneously; providing a method and compositions for re-sequencing predetermined or determinable regions of a genome in order to detect sequence variation; providing a method for generating sets of labeled oligonucleotide tags containing sequence information about a polynucleotide; and providing a method for simultaneously generating signature sequences for a population of polynucleotides or sequencing templates.

The invention achieves these and other objectives in its various aspects and embodiments as disclosed below. Preferably, the method of the invention is carried out with the following steps: (i) attaching an oligonucleotide tag from a repertoire of tags to each polynucleotide of the population to form tag-polynucleotide conjugates such that substantially every different polynucleotide has a different oligonucleotide tag attached; (ii) generating a size ladder of polynucleotide fragments for each tag-polynucleotide conjugate, each polynucleotide fragment of the same size ladder having an end and the same oligonucleotide tag as every other polynucleotide fragment of the size ladder; (iii) separating the polynucleotide fragments into size classes; (iv) labeling the oligonucleotide tag of each polynucleotide fragment according to the identity of one or more nucleotides at the end of such polynucleotide fragment; (v) copying the oligonucleotide tags of each polynucleotide fragment of each size class; and (vi) separately hybridizing labeled oligonucleotide tags of each size class with their respective complements under stringent hybridization conditions, the respective complements being attached as populations of substantially identical oligonucleotides in spatially discrete and addressable regions on one or more solid phase supports, and the respective signature sequences being determined by the sequence of labels associated with each spatially discrete and addressable region of the one or more solid phase supports. As illustrated further below, the ordering of the steps of separating, labeling, and copying may vary depending on the particular embodiment. The invention includes materials and kits for carrying out the above method.

The present invention overcomes shortcomings in the art by providing a simpler and more convenient means for generating size ladders of polynucleotide fragments and for copying tags for specific hybridization to one or more arrays of tag complements. In particular, a preferred embodiment of the invention not only reduces the burden of template preparation by the use of olignucleotide tags, but also allows for read-outs of full signatures in the time it takes to perform a single hybridization reaction by the simultaneous hybridization of tags of different size classes to separate arrays.

### BRIEF DESCRIPTION OF THE DRAWINGS

FIG. 1a illustrates the general scheme of the invention wherein tagged polynucleotides are processed to form size ladders of polynucleotide fragments after which oligonucleotide tags are copied and specifically hybridized to one or more hybridization arrays.

FIG. 1b illustrates an embodiment of the invention wherein a sample of tag-polynucleotide conjugates are processed to produce a mixture of size classes of polynucleotide fragments which are then physically separated by size; their tags are amplified and labeled; and finally, they are applied simultaneously to a plurality of microarrays for hybridization with tag complements.

FIGS. 2a through 2g illustrate a scheme for generating size ladders using a type IIs restriction endonuclease and for identifying pairs of nucleotides by ligation of an adaptor to the end of each member of each size class to form signature sequences.

FIGS. 3a and 3b illustrate a scheme for generating size ladders using a combination of type IIs restriction endonucleases and primers having 3′ ends with degenerate nucleotides forming duplexes up to five nucleotides into the polynucleotide fragment. Individual nucleotides are identified by extending the primers by a single dideoxynucleotide.

FIGS. 4a and 4b illustrate a scheme for generating size ladders by extending a primer by ligation of random 6-mers on a polynucleotide template and for identifying individual nucleotides by polymerase extension.

FIG. 5 illustrates an apparatus for hybridizing labeled tags to an array of microbeads.

### DEFINITIONS

“Complement” or “tag complement” as used herein in reference to oligonucleotide tags refers to an oligonucleotide to which an oligonucleotide tag specifically hybridizes to form a perfectly matched duplex or triplex. In embodiments where specific hybridization results in a triplex, the oligonucleotide tag may be selected to be either double stranded or single stranded. Thus, where triplexes are formed, the term “complement” is meant to encompass either a double stranded complement of a single stranded oligonucleotide tag or a single stranded complement of a double stranded oligonucleotide tag.

The term “oligonucleotide” as used herein includes linear oligomers of natural or modified monomers or linkages, including deoxyribonucleosides, ribonucleosides, anomeric forms thereof, peptide nucleic acids (PNAs), and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 34, to several tens of monomeric units, e.g. 40-60. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. Usually oligonucleotides of the invention comprise the four natural nucleotides; however, they may also comprise non-natural nucleotide analogs. It is clear to those skilled in the art when oligonucleotides having natural or non-natural nucleotides may be employed, e.g. where processing by enzymes is called for, usually oligonucleotides consisting of natural nucleotides are required.

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex. Conversely, a “mismatch” in a duplex between a tag and an oligonucleotide means that a pair or triplet of nucleotides in the duplex or triplex fails to undergo Watson-Crick and/or Hoogsteen and/or reverse Hoogsteen bonding.

As used herein, “nucleoside” includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the only proviso that they are capable of specific hybridization. Such analogs include synthetic nucleosides designed to enhance binding properties, reduce complexity, increase specificity, and the like.

As used herein “sequence determination” or “determining a nucleotide sequence” in reference to polynucleotides includes determination of partial as well as full sequence information of the polynucleotide. That is, the term includes sequence comparisons, fingerprinting, and like levels of information about a target polynucleotide, as well as the express identification and ordering of nucleosides, usually each nucleoside, in a target polynucleotide. The term also includes the determination of the identity, ordering, and locations of one, two, or three of the four types of nucleotides within a target polynucleotide. For example, in some embodiments sequence determination may be effected by identifying the ordering and locations of a single type of nucleotide, e.g. cytosines, within the target polynucleotide “CATCGC . . . ” so that its sequence is represented as a binary code, e.g. “100101 . . . ” for “C-(not C)-(not C)-C-(not C)-C . . . ” and the like.

As used herein “signature sequence” means a sequence of nucleotides derived from a polynucleotide such that the ordering of nucleotides in the signature is the same as their ordering in the polynucleotide and the sequence contains sufficient information to identify the polynucleotide in a population. Signature sequences may consist of a segment of consecutive nucleotides (such as, (a,c,g,t,c) of the polynucleotide “acgtcggaaatc”), or it may consist of a sequence of every second nucleotide (such as, (c,t,g,a,a,) of the polynucleotide “acgtcggaaatc”), or it may consist of a sequence of nucleotide changes (such as, (a,c,g,t,c,g,a,t,c) of the polynucleotide “acgtcggaaatc”), or like sequences.

As used herein, the term “complexity” in reference to a population of polynucleotides means the number of different species of polynucleotide present in the population.

As used herein, “amplicon” means the product of an amplification reaction. That is, it is a population of polynucleotides, usually double stranded, that are replicated from one or more starting sequences. The one or more starting sequences may be one or more copies of the same sequence, or it may be a mixture of different sequences. Preferably, amplicons are produced either in a polymerase chain reaction (PCR) or by replication in a cloning vector.

As used herein, “addressable” in reference to tag complements means that the nucleotide sequence, or perhaps other physical or chemical characteristics, of a tag complement can be determined from its address, i.e. a one-to-one correspondence between the sequence or other property of the tag complement and a spatial location on, or characteristic of, the solid phase support to which it is attached. Preferably, an address of a tag complement is a spatial location, e.g. the planar coordinates of a particular region containing copies of the tag complement. However, tag complements may be addressed in other ways too, e.g. by microparticle size, shape, color, frequency of micro-transponder, or the like, e.g. Chandler et al, PCT publication WO 97/14028.

As used herein, “ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically.

As used herein, “microarray” refers to a solid phase support having a planar surface, which carries an array of nucleic acids, each member of the array comprising identical copies of an oligonucleotide or polynucleotide immobilized to a fixed region, which does not overlap with those of other members of the array. Typically, the oligonucleotides or polynucleotides are single stranded and are covalently attached to the solid phase support. The density of non-overlapping regions containing nucleic acids in a microarray is typically greater than 100 per cm², and more preferably, greater than 1000 per cm². Microarray technology is reviewed in the following references: Schena et al, Trends in Biotechnology, 16: 301-306 (1998); Southern, Current Opin. Chem. Biol., 2: 404-410 (1998); Nature Genetics Supplement, 21: 1-60 (1999).

### DETAILED DESCRIPTION OF THE INVENTION

The invention provides a method of simultaneously sequencing polynucleotides in a complex mixture by using oligonucleotide tags to shuttle sequence information obtained from the polynucleotides to discrete spatially addressable sites on one or more solid phase supports, such as a microarray or a collection of microarrays. After oligonucleotide tags specifically hybridize to their respective complements at the spatially addressable sites on the solid phase supports, sequence information is conveyed by the signals generated by labels on the tags. When the same solid phase support is employed for all hybridization reactions, such as a microbead array, signature sequences are generated by carrying out successive cycles of hybridizing, detecting, and washing, with sets of labeled tags derived from different size classes of fragments. When a plurality of identical solid phase supports are employed, such as a collection of microarrays, signature sequences may be obtained simultaneously by separate hybridizations to the plurality of solid phase supports in order to generate simultaneously signature sequences of the polynucleotides in the mixture. In each of the separate hybridizations, only labeled tags from a single size class of polynucleotide fragment are present. Thus, a set of signals produced at the location on the plurality of different microarrays gives a read-out of a complete signature sequence of one of the polynucleotides of the mixture.

In accordance with the invention, polynucleotides of a complex mixture are conjugated to oligonucleotide tags to form a population of tag-polynucleotide conjugates, as described in Brenner et al, U.S. Pat. No. 5,846,719, and Brenner et al, Proc. Natl. Acad. Sci., 97: 1665-1670 (2000), which are incorporated by reference. By selecting a repertoire of tags having a substantially larger number of distinct species than those of the population of polynucleotides, a sample of conjugates can be selected which is large enough so that all of the different species of polynucleotide are included, but which is also small enough so that substantially every polynucleotide will have a unique tag. Preferably, the sample size is a few percent, e.g. less than 10 percent, of the size of the tag repertoire.

An important feature of the invention is the generation of a size ladder of polynucleotide fragments for each tag-polynucleotide conjugate of the sample. As used herein, the term “size ladder” in reference to a tag-polynucleotide conjugate means a series of polynucleotide fragments generated from the tag-polynucleotide conjugate, wherein each polynucleotide fragment of the same size ladder has the same tag attached and wherein the lengths of each of the polynucleotide fragments within a size ladder differ from one another by a predetermined number of nucleotides. That is, the a size ladder may be generated by removing predetermined numbers of nucleotides from a tag-polynucleotide conjugate, or it may be generated by extending a primer a predetermined number of nucleotides on a template derived from a tag-polynucleotide conjugate. For example, in a simple case, a size ladder is generated by successively removing a single nucleotide from the end of the polynucleotide of a tag-polynucleotide conjugate, so that the size ladder consists of a series of polynucleotide fragments each differing in length from its closest neighbor by one nucleotide. However, it is not necessary that the size classes of a size ladder differ in length by multiples of a constant number of nucleotides. A size ladder may consist of any series of polynucleotide fragments whose ends terminate at any of a collection of nucleotide positions that are the same for all the different tag-polynucleotide conjugates of a mixture. The important features is that the differences in fragment sizes within a size ladder not vary from fragment to fragment so that a correspondence exists between the signature sequence generated and the polynucleotide it is derived from. Preferably, the size differences between fragments of a size ladder are predetermined and are the same for all the tag-polynucleotide conjugates.

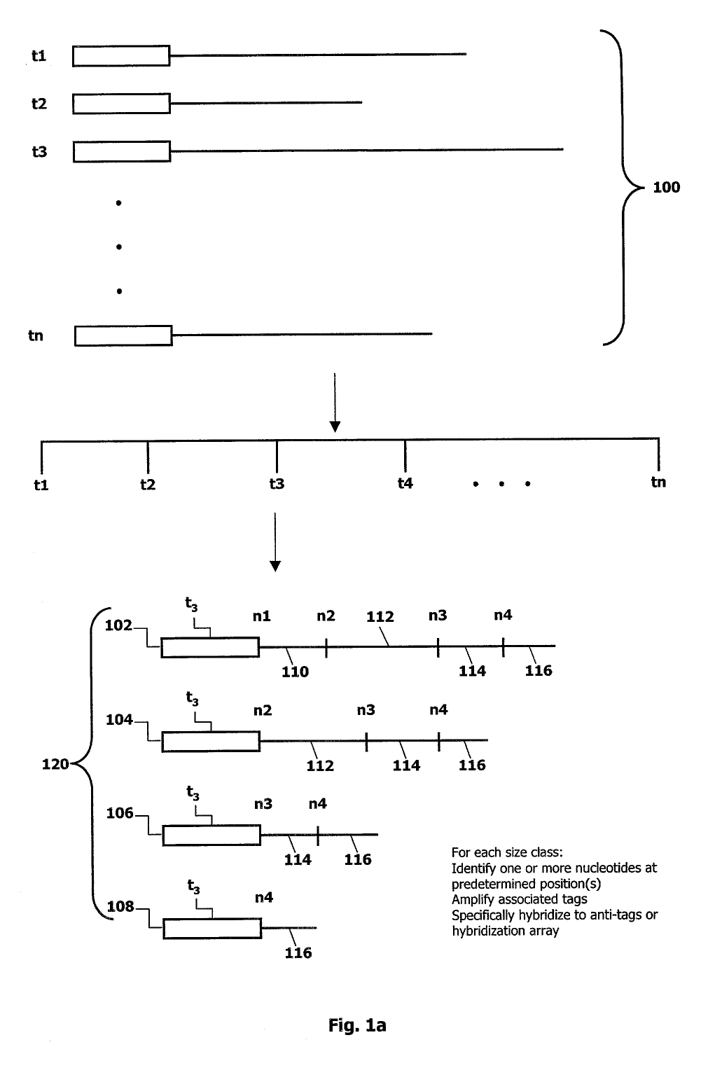

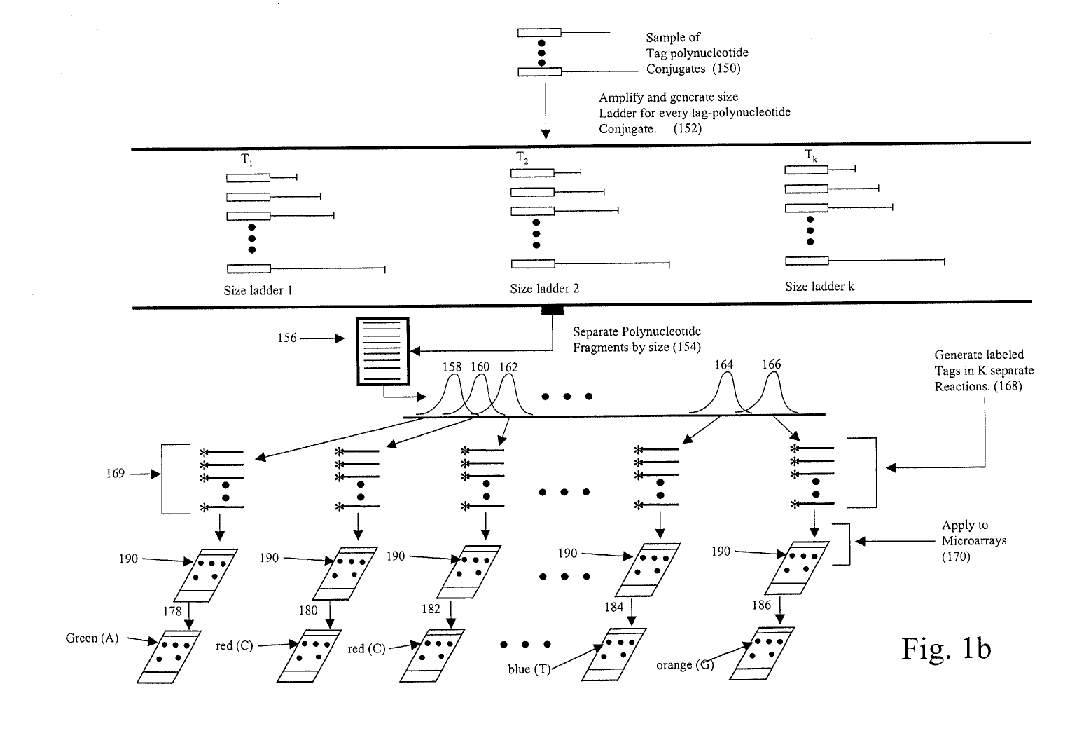

The concept of size ladder is illustrated in FIG. 1a. Sample (100) of tag-polynucleotide conjugates, t₁, t₂, t₃, to tₙ, is operated on to produce a predetermined number of size classes of polynucleotide fragments for each conjugate, e.g. (102), (104), (106), and (108) as shown for conjugate t₃. In this example, the size ladder (120) is generated by removing fragment (110) between nucleotide positions n₁ and n₂ from conjugate (102) to form conjugate (104), removing fragment (112) between nucleotide positions n₂ and n₃ from conjugate (104) to form conjugate (106), and removing fragment (114) between nucleotide positions n₃ and n₄ from conjugate (106) to form conjugate (108). All of the conjugates (102) through (108) have the same oligonucleotide tag, t₃. Thus, in this example, after generation of the size ladder, in conjugate (102), tag t₃ is immediately adjacent to the nucleotide at position n₁; in conjugate (104), tag t₃ is immediately adjacent to the nucleotide at position n₂; in conjugate (106), tag t₃ is immediately adjacent to the nucleotide at position n₃; and in conjugate (108), tag t₃ is immediately adjacent to the nucleotide at position n₄. Thus, if the illustrated embodiment was designed to create a label on tag t₃ that indicated the identity of the immediately adjacent nucleotide, then after amplification, labeling, and four cycles of hybridization, detection, and washing, a signature consisting of the identities of the nucleotides at positions n₁, n₂, n₃, and n₄ would be generated.

Clearly, size ladders can be generated in several different ways and the positions at which nucleotides are identified in the different size classes of a size ladder can vary also. For example, in FIG. 1, the illustrated size ladder can be generated by successively removing fragments (116), (114), and (112), with a label being attached to the respective tags which identifies one or more nucleotides at positions in the fragment distal-most to the tag. In this case, the nucleotide immediately adjacent to the tag is the same in all size classes and the nucleotides of the distal-most position vary.

The number of size classes in a size ladder can vary widely; however, preferably, the number is large enough to permit unique signatures to be generated for the polynucleotides of the population being analyzed. Other factors affecting the selection of the number of size classes include the means for generating the size classes (i.e., the ability to produce well defined size classes may depend on the sizes and/or complexity of the tag-polynucleotide conjugate mixture), and for embodiments requiring physical separation, the means for carrying out the separation may have limited resolving power for very complex mixtures of tag-polynucleotide conjugates. Preferably, the number of size classes in a size ladder is at least 12; and more preferably, at least 16. Still more preferably, a size ladder as between 12 and 100 size classes. Still more preferably, a size ladder has between 12 and 60 size classes; and most preferably, it has between 16 and 36 size classes.

The use of size ladders in a preferred embodiment of the invention is further illustrated in FIG. 1b. A sample (150) of tag-polynucleotide conjugates is amplified and size ladders are generated by extending primers predetermined amounts along tag-polynucleotide templates (in a manner exemplified below) to give a mixture consisting of multiple copies of each size class of polynucleotide fragment of each ladder. The mixture is then separated (154) by a conventional DNA separation technique such as preparative gel electrophoresis or HPLC. Preferably, in this embodiment, polynucleotide fragments are separated into size classes using denaturing HPLC, which is more amenable to automation than preparative gel electrophoresis, e.g. as disclosed in the following references: Devaney et al, Application Notes Nos. 103, 107 and 110 (Transgenomic, Inc., Omaha, Nebr.); Huber et al, Anal. Chem. 67: 578-585 (1995); Dickman et al, Anal. Biochem., 284: 164-167 (2000); Oefner et al, Anal. Biochem., 223: 39-46 (1994). Preferably, the separation technique produces peaks (158), (160), (162), (164), (166), and the like, of well-separated and isolatable size classes of polynucleotide fragments. Peaks (158), (160), (162), (164), (166), and so on, are eluted from the separation column and placed into separate reaction vessels (168) where the tags of the fragments are amplified and labeled according to the nucleotide being identified. As illustrated below, such identification can be accomplished in several ways, including identification based on single nucleotide extension of a primer using a DNA polymerase or identification based on the ligation of adaptors to the polynucleotide fragments. Labeled tags (169) from peaks (158), (160), (162), (164), (166), and so on, are then applied to their respective microarrays (178), (180), (182), (184), (186), and so on, where they specifically hybridize to the tag complements on the microarrays under stringent hybridization conditions. After washing, signatures are determined by measuring the signals generated at the same addresses of the different microarrays, illustrated by (190) in FIG. 1b. In some embodiments, this may be accomplished by providing different colored fluorescent dyes for each of the different nucleotides, A, C, G, and T, as illustrated in FIG. 1b, where a green signal represents an “A”, a red signal represents a “C”, a blue signal represents a “T”, and an orange signal represents a “G,” to give a signature of “GT . . . CCA” (assuming that the 5′-most nucleotides are associated with the smaller fragments). In other embodiments, a single dye may used and nucleotide identity may be determined by providing four separate hybridizations for each size class, wherein the read-out from the same address from each of the four microarrays is one positive signal and three negative signals, such that a positive signal at microarray 1 indicates “a”, a positive signal at microarray 2 indicates “c”, and so on.

### Formation of Tag-Polynucleotide Conjugates and Sampling

An important feature of the invention is the use of oligonucleotide tags consisting of oligonucleotides selected from a minimally cross-hybridizing set of oligonucleotides, or assembled from oligonucleotide subunits selected from a minimally cross-hybridizing set of oligonucleotides. Construction of such minimally cross-hybridizing sets are disclosed in Brenner et al, U.S. Pat. No. 5,846,719, and Brenner et al, Proc. Natl. Acad. Sci., 97: 1665-1670 (2000), which references are incorporated by reference. The sequences of oligonucleotides of a minimally cross-hybridizing set differ from the sequences of every other member of the same set by at least two nucleotides. Thus, each member of such a set cannot form a duplex (or triplex) with the complement of any other member with less than two mismatches. Preferably, perfectly matched duplexes of tags and tag complements of the same minimally cross-hybridizing set have approximately the same stability, especially as measured by melting temperature. Complements of oligonucleotide tags, referred to herein as “tag complements,” may comprise natural nucleotides or non-natural nucleotide analogs. Oligonucleotide tags when used with their corresponding tag complements provide a means of enhancing specificity of hybridization.

Minimally cross-hybridizing sets of oligonucleotide tags and tag complements may be synthesized either combinatorially or individually depending on the size of the set desired and the degree to which cross-hybridization is sought to be minimized (or stated another way, the degree to which specificity is sought to be enhanced). For example, a minimally cross-hybridizing set may consist of a set of individually synthesized 10-mer sequences that differ from each other by at least 4 nucleotides, such set having a maximum size of 332, when constructed as disclosed in Brenner et al, International patent application PCT/US96/09513. Alternatively, a minimally cross-hybridizing set of oligonucleotide tags may also be assembled combinatorially from subunits which themselves are selected from a minimally cross-hybridizing set. For example, a set of minimally cross-hybridizing 12-mers differing from one another by at least three nucleotides may be synthesized by assembling 3 subunits selected from a set of minimally cross-hybridizing 4-mers that each differ from one another by three nucleotides. Such an embodiment gives a maximally sized set of 9³, or 729, 12-mers.

When synthesized combinatorially, an oligonucleotide tag preferably consists of a plurality of subunits, each subunit consisting of an oligonucleotide of 3 to 9 nucleotides in length wherein each subunit is selected from the same minimally cross-hybridizing set. In such embodiments, the number of oligonucleotide tags available depends on the number of subunits per tag and on the length of the subunits.

Preferably, tag complements are synthesized on the surface of a solid phase support, such as a microscopic bead or a specific location on an array of synthesis locations on a single support, such that populations of identical, or substantially identical, sequences are produced in specific regions. That is, the surface of each support, in the case of a bead, or of each region, in the case of an array, is derivatized by copies of only one type of tag complement having a particular sequence. The population of such beads or regions contains a repertoire of tag complements each with distinct sequences. As used herein in reference to oligonucleotide tags and tag complements, the term “repertoire” means the total number of different oligonucleotide tags or tag complements. A repertoire may consist of a set of minimally cross-hybridizing set of oligonucleotides that are individually synthesized, or it may consist of a concatenation of oligonucleotides each selected from the same set of minimally cross-hybridizing oligonucleotides. In the latter case, the repertoire is preferably synthesized combinatorially.

When tag complements are attached to or synthesized on microbeads, a wide variety of solid phase materials may be used with the invention, including microbeads made of controlled pore glass (CPG), highly cross-linked polystyrene, acrylic copolymers, cellulose, nylon, dextran, latex, polyacrolein, and the like, disclosed in the following exemplary references: Meth. Enzymol., Section A, pages 11-147, vol. 44 (Academic Press, New York, 1976); U.S. Pat. Nos. 4,678,814; 4,413,070; and 4,046;720; and Pon, Chapter 19, in Agrawal, editor, Methods in Molecular Biology, Vol. 20, (Humana Press, Totowa, N.J., 1993). Microbead supports further include commercially available nucleoside-derivatized CPG and polystyrene beads (e.g. available from Applied Biosystems, Foster City, Calif.); derivatized magnetic beads; polystyrene grafted with polyethylene glycol (e.g., TentaGel™, Rapp Polymere, Tubingen Germany); and the like. Generally, the size and shape of a microbead is not critical; however, microbeads in the size range of a few, e.g. 1-2, to several hundred, e.g. 200-1000 μm diameter are preferable, as they facilitate the construction and manipulation of large repertoires of oligonucleotide tags with minimal reagent and sample usage. Preferably, glycidal methacrylate (GMA) beads available from Bangs Laboratories (Carmel, Ind.) are used as microbeads in the invention. Such microbeads are useful in a variety of sizes and are available with a variety of linkage groups for synthesizing tags and/or tag complements.

Preferably, prior to generating size ladders of polynucleotide fragments, a set of tag-polynucleotide conjugates is produced such that substantially all different polynucleotides have different tags attached. This condition is achieved by employing a repertoire of tags substantially greater than the population of polynucleotides and by taking a sufficiently small sample of tagged polynucleotides from the full ensemble of tagged polynucleotides.

Sets containing several hundred to several thousands, or even several tens of thousands, of oligonucleotides may be synthesized directly by a variety of parallel synthesis approaches, e.g. as disclosed in Frank et al, U.S. Pat. No. 4,689,405; Frank et al., Nucleic Acids Research, 11: 4365-4377 (1983); Matson et al, Anal. Biochem., 224: 110-116 (1995); Fodor et al., International application PCT/US93/04145; Pease et al, Proc. Natl. Acad. Sci., 91: 5022-5026 (1994); Southern et al, J. Biotechnology, 35: 217-227 (1994), Brennan, International application PCT/US94/05896; Lashkari et al, Proc. Natl. Acad. Sci., 92: 7912-7915 (1995); or the like.

Preferably, tag complements in mixtures, whether synthesized combinatorially or individually, are selected to have similar duplex or triplex stabilities to one another so that perfectly matched hybrids have similar or substantially identical melting temperatures. This permits mis-matched tag complements to be more readily distinguished from perfectly matched tag complements in the hybridization steps, e.g. by washing under stringent conditions. For combinatorially synthesized tag complements, minimally cross-hybridizing sets may be constructed from subunits that make approximately equivalent contributions to duplex stability as every other subunit in the set. Guidance for carrying out such selections is provided by published techniques for selecting optimal PCR primers and calculating duplex stabilities, e.g. Rychlik et al, Nucleic Acids Research, 17: 8543-8551 (1989) and 18: 6409-6412 (1990); Breslauer et al, Proc. Natl. Acad. Sci., 83: 3746-3750 (1986); Wetmur, Crit. Rev. Biochem. Mol. Biol., 26: 227-259 (1991); and the like. A minimally cross-hybridizing set of oligonucleotides can be screened by additional criteria, such as GC-content, distribution of mismatches, theoretical melting temperature, and the like, to form a subset which is also a minimally cross-hybridizing set.

The oligonucleotide tags of the invention and their complements are conveniently synthesized on an automated DNA synthesizer, e.g. an Applied Biosystems, Inc. (Foster City, Calif.) model 392 or 394 DNA/RNA Synthesizer, using standard chemistries, such as phosphoramidite chemistry, e.g. disclosed in the following references: Beaucage and Iyer, Tetrahedron, 48: 2223-2311 (1992); Molko et al, U.S. Pat. No. 4,980,460; Koster et al, U.S. Pat. No. 4,725,677; Caruthers et al, U.S. Pat. Nos. 4,415,732; 4,458,066; and 4,973,679; and the like. Preferably, oligonucleotide tags of the invention are assembled enzymatically as disclosed by Brenner et al, International patent application PCT/US00/20639.

Tag-polynucleotide conjugates are conveniently formed by inserting the set of polynucleotides being analyzed into a vector containing a library of oligonucleotide tags, as shown below (SEQ ID NO: 1).

```
Formula I
        Left Primer Bsp 120I
5′-AGAATTCGGGCCTTAATTAA ↓
5′- AGAATTCGGGCCTTAATTAA- [⁶(A,C,G,T)₄]-GGGCCC-
        TCTTAAGCCCGGAATTAATT- [⁶(T,G,C,A)₄]-CCCGGG-
              ↑ ↑
          Eco RI Pac I
                                                              Bbs I Bam HI
                                                                ↓ ↓
                                          -GCATAAGTCTTCXXX . . . XXXGGATCCGAGTGAT -3′
                                          -CGTATTCAGAAGXXX . . . XXXCCTAGGCTCACTA
                                                                                            XXXXXCCTAGGCTCACTA-5′
                                                                                                  Right Primer
```

The flanking regions of the oligonucleotide tag may be engineered to contain restriction sites, as exemplified above, for convenient insertion into and excision from cloning vectors. Optionally, the right or left primers may be synthesized with a biotin attached (using conventional reagents, e.g. available from Clontech Laboratories, Palo Alto, Calif.) to facilitate purification after amplification and/or cleavage. Preferably, for making tag-fragment conjugates, the above library is inserted into a conventional cloning vector, such a pUC 19, or the like. Optionally, the vector containing the tag library may contain a “stuffer” region, “XXX . . . XXX,” which facilitates isolation of fragments fully digested with, for example, Bam HI and Bbs I.

The steps of inserting cDNAs into such a vector are illustrated in FIGS. 6a and 6b. First, mRNA (300) is extracted from a cell or tissue source of interest using conventional techniques and is converted into cDNA (309) with ends appropriate for inserting into vector (316). Preferably, primer (302) having a 5′ biotin (305) and poly(dT) region (306) is annealed to mRNA strands (300) so that the first strand of cDNA (309) is synthesized with a reverse transcriptase in the presence of the four deoxyribonucleoside triphosphates. Preferably, 5-methyldeoxycytidine triphosphate is used in place of deoxycytosine triphosphate in the first strand synthesis, so that cDNA (309) is hemi-methylated, except for the region corresponding to primer (302). This allows primer (302) to contain a non-methylated restriction site for releasing the cDNA from a support. The use of biotin in primer (302) is not critical to the invention and other molecular capture techniques, or moieties, can be used, e.g. triplex capture, or the like. Region (303) of primer (302) preferably contains a sequence of nucleotides that results in the formation of restriction site r₂ (304) upon synthesis of the second strand of cDNA (309). After isolation by binding the biotinylated cDNAs to streptavidin supports, e.g. Dynabeads M-280 (Dynal, Oslo, Norway), or the like, cDNA (309) is preferably cleaved with a restriction endonuclease which is insensitive to hemimethylation (of the C's) and which recognizes site r₁ (307). Preferably, r₁ is a four-base recognition site, e.g. corresponding to Dpn II, or like enzyme, which ensures that substantially all of the cDNAs are cleaved and that the same defined end is produced in all of the cDNAs. After washing, the cDNAs are then cleaved with a restriction endonuclease recognizing r₂, releasing fragment (308) which is purified using standard techniques, e.g. ethanol precipitation, polyacrylamide gel electrophoresis, or the like. After resuspending in an appropriate buffer, fragment (308) is directionally ligated into vector (316), which carries tag (310) and a cloning site with ends (312) and (314). Preferably, vector (316) is prepared with a “stuffer” fragment in the cloning site to aid in the isolation of a fully cleaved vector for cloning.

After formation of a library of tag-cDNA conjugates, a sample of host cells is usually plated to determine the number of recombinants per unit volume of culture medium. The size of sample taken for further processing preferably depends on the size of tag repertoire used in the library construction, as discussed above. Preferably, tag-cDNA conjugates are carried in vector (330) which comprises the following sequence of elements: first primer binding site (332), restriction site r₃ (334), oligonucleotide tag (336), junction (338), cDNA (340), restriction site r₄ (342), and second primer binding site (344). After a sample is taken of the vectors containing tag-cDNA conjugates the following steps are implemented: The tag-cDNA conjugates may be amplified from vector (330) by use of biotinylated primer (348) and labeled primer (346) in a conventional polymerase chain reaction (PCR) in the presence of 5-methyldeoxycytidine triphosphate, after which the resulting amplicon is isolated by streptavidin capture. Restriction site r₃ preferably corresponds to a rare-cutting restriction endonuclease, such as Pac I, Not I, Fse I, Pme I, Swa I, or the like, which permits the captured amplicon to be release from a support with minimal probability of cleavage occurring at a site internal to the cDNA of the amplicon.

An important aspect of the invention is that substantially all different DNA sequences have different tags attached. This condition is brought about by taking only a sample of the full ensemble of tag-polynucleotide conjugates for analysis. (It is acceptable that identical polynucleotides have different tags, as it merely results in the same polynucleotide being analyzed twice.) Such sampling can be carried out either overtly—for example, by taking a small volume from a larger mixture—after the tags have been attached to the DNA sequences; it can be carried out inherently as a secondary effect of the techniques used to process the DNA sequences and tags; or sampling can be carried out both overtly and as an inherent part of processing steps.

If a sample of n tag-DNA sequence conjugates are randomly drawn from a reaction mixture—as could be effected by taking a sample volume, the probability of drawing conjugates having the same tag is described by the Poisson distribution, P(r)=e^{-λ}(λ)^{r}/r, where r is the number of conjugates having the same tag and λ=np, where p is the probability of a given tag being selected. If n=10⁶ and p=1/(1.67×10⁷) (for example, if eight 4-base words described in Brenner et al were employed as tags), then λ=0.0149 and P(2)=1.13×10⁻⁴. Thus, a sample of one million molecules gives rise to an expected number of doubles well within the preferred range. Such a sample is readily obtained by serial dilutions of a mixture containing tag-fragment conjugates.

As used herein, the term “substantially all” in reference to attaching tags to molecules, especially polynucleotides, is meant to reflect the statistical nature of the sampling procedure employed to obtain a population of tag-molecule conjugates essentially free of doubles. Preferably, at least ninety-five percent of the DNA sequences have unique tags attached.

Preferably, DNA sequences are conjugated to oligonucleotide tags by inserting the sequences into a conventional cloning vector carrying a tag library. For example, cDNAs may be constructed having a Bsp 120 I site at their 5′ ends and after digestion with Bsp 120 I and another enzyme such as Sau 3A or Dpn II may be directionally inserted into a pUC19 carrying the tags of Formula I to form a tag-cDNA library, which includes every possible tag-cDNA pairing. A sample is taken from this library for analysis. Sampling may be accomplished by serial dilutions of the library, or by simply picking plasmid-containing bacterial hosts from colonies. After amplification, the tag-cDNA conjugates may be excised from the plasmid. The sample of conjugates is used to generate a size ladder of polynucleotide fragments.

Selection of a tag repertoire to be used with the invention is a matter of design choice which may be influenced by several factors, including the number of signature sequences to be determined per operation, i.e. the throughput, the duration of hybridization reaction(s), tolerance to non-specific hybridizations, the number of polynucleotides being analyzed per operation, the size of tag desired, the size of hybridization array available, tolerance to “doubles,” composition of words, and the like. Preferably, a repertoire of tags is selected that is produced by combinatorial synthesis of words, e.g. as disclosed by Brenner et al, Proc. Natl. Acad. Sci., 97: 1665-1670 (2000). This permits the efficient synthesis of a large number of tags with similar properties. Preferably, a repertoire of tags consists of between about 5×10⁴ and about 2×10⁶ tags of different nucleotide sequences. In other words, the size of the repertoire is preferably between about 5×10⁴ and about 5×10⁶. For samples of tag-polynucleotide conjugates in the range of between about one and about ten percent of the repertoire size, this results in hybridization reactions of mixtures having complexities in the range of from 50 to 5×10⁵ species. That is, such parameter selections require hybridization reactions that involve the formation of a number of detectable duplexes between about 500 and about 5×10⁵. Preferably, as used here, “detectable duplex” means that the signal-to-noise ratio of a signal collected from a labeled tag at a hybridization site is at least 2; more preferably, it is at least 3.

The specificity of the hybridization reactions of tags and tag complements may be increased by selecting words that have a larger number of mismatches between non-perfectly matched sequences. Preferably, tags of the present invention are constructed from 6-mer words selected from the set listed in Table I. Each word of this set forms a duplex with at least four mismatches with the complements of any other word of the same set. In further preference, tags used in the invention are constructed from a concatenation of four words selected from the set of Table I. Preferably, each word is separated from its neighboring word by a “spacer” nucleotide so that the preferred words have the form:
. . . wwwwwwnwwwwwwnwwwwwwnwwwwww . . .

where “w” designates a nucleotide of a word and “n” designates a “spacer” nucleotide. Tags with such a structure give rise to a repertoire size of 32⁴, or 1,048,576 tags. The sequences and melting temperatures of the tags generated by such words are readily listed using computer programs such as that disclosed in Appendix 1. For the set of words of Table I, distributions of melting temperatures were calculated for tags forming perfectly matched duplexes, tags forming duplexes with a mismatch in the 3′-most word, and tags forming duplexes with a mismatch in the 5′-most word (i.e. the most stable of the single word mismatches). The results are shown in Appendix 2, and demonstrate that with such a set of tags, wash temperatures can be selected that above which perfectly matched tag duplexes are stable and below which all tag duplexes containing mismatches are unstable and will dissociate.

TABLE I
Minimally cross-hybridizing set of 6-mers used to form
27-mer tags having one nucleotide spacers between words
(Below, 1, 2, 3, and 4, stand for a, c, g, and t, respectively)

| | | | |
|---|---|---|---|
| 121243 | 212431 | 331441 | 413241 |
| 124312 | 213124 | 334114 | 414132 |
| 133142 | 221414 | 341313 | 421331 |
| 141432 | 224141 | 342424 | 422442 |
| 142341 | 242112 | 343131 | 423113 |
| 143214 | 243443 | 344242 | 424224 |
| 144123 | 313412 | 411423 | 432121 |
| 211342 | 314321 | 412314 | 433434 |

Oligonucleotide tags generated in accordance with the invention can be labeled in a variety of ways, including the direct or indirect attachment of radioactive moieties, fluorescent moieties, calorimetric moieties, chemiluminescent moieties, and the like. Many comprehensive reviews of methodologies for labeling DNA provide guidance applicable to generating labeled oligonucleotide tags of the present invention. Such reviews include Haugland, Handbook of Fluorescent Probes and Research Chemicals, Sixth Edition (Molecular Probes, Inc., Eugene, 2001); Keller and Manak, DNA Probes, 2nd Edition (Stockton Press, New York, 1993); Eckstein, editor, Oligonucleotides and Analogues: A Practical Approach (IRL Press, Oxford, 1991); Wetmur, Critical Reviews in Biochemistry and Molecular Biology, 26: 227-259 (1991); and the like. Many more particular methodologies applicable to the invention are disclosed in the following sample of references: Fung et al., U.S. Pat. No. 4,757,141; Hobbs, Jr., et al. U.S. Pat. No. 5,151,507; Cruickshank, U.S. Pat. No. 5,091,519; (synthesis of functionalized oligonucleotides for attachment of reporter groups); Jablonski et al, Nucleic Acids Research, 14: 6115-6128 (1986)(enzyme-oligonucleotide conjugates).

Selection of fluorescent dyes and means for attaching or incorporating them into DNA strands is well known, e.g. Matthews et al, Anal. Biochem., Vol 169, pgs. 1-25 (1988); Haugland, Handbook of Fluorescent Probes and Research Chemicals (Molecular Probes, Inc., Eugene, 2001); Keller and Manak, DNA Probes, 2nd Edition (Stockton Press, New York, 1993); and Eckstein, editor, Oligonucleotides and Analogues: A Practical Approach (IRL Press, Oxford, 1991); Wetmur, Critical Reviews in Biochemistry and Molecular Biology, 26: 227-259 (1991); Ju et al, Proc. Natl. Acad. Sci., 92: 4347-4351 (1995) and Ju et al, Nature Medicine, 2: 246-249 (1996); and the like. Preferably, one or more fluorescent dyes are used as labels for the oligonucleotide tags, e.g. as disclosed by Menchen et al, U.S. Pat. No. 5,188,934 (4,7-dichlorofluorscein dyes); Begot et al, U.S. Pat. No. 5,366,860 (spectrally resolvable rhodamine dyes); Lee et al, U.S. Pat. No. 5,847,162 (4,7-dichlororhodamine dyes); Khanna et al, U.S. Pat. No. 4,318,846 (ether-substituted fluorescein dyes); Lee et al, U.S. Pat. No. 5,800,996 (energy transfer dyes); Lee et al, U.S. Pat. No. 5,066,580 (xanthene dyes): Mathies et al, U.S. Pat. No. 5,688,648 (energy transfer dyes); and the like. As used herein, the term “fluorescent signal generating moiety” means a signaling means which conveys information through the fluorescent absorption and/or emission properties of one or more molecules. Such fluorescent properties include fluorescence intensity, fluorescence life time, emission spectrum characteristics, energy transfer, and the like.

### Hybridization Arrays

Labeled oligonucleotide tags of the invention are detected by specifically hybridizing them to a spatially addressable array of complementary sequences. Preferably such arrays are microarrays, so that the quantities of reactants, e.g. labeled tags, or the like, and the volumes of reagents in the hybridization reaction may be minimized. Such arrays include arrays of microbeads as disclosed by Brenner et al, International patent application PCT/US98/11224, or microarrays which contain a regularly spaced planar array of hybridization sites, e.g. as disclosed in the references cited below. When microbead arrays are employed, the number of microbead making up the array are preferably at least five times the number of tags in the repertoire being used. This ensures that with high probability the array contains at least one microbead for every tag in the repertoire. Thus, if the size of the tag repertoire is 10, and if the microbead array contains 5×10⁵ microbeads, then with probability of 99% every tag of the repertoire will be represented in the microbead array. Preferably, planar microarrays made by conventional technologies are employed. Such microarrays may be manufactured by several alternative techniques, such as photo-lithographic optical methods, e.g. Pirrung et al, U.S. Pat. No. 5,143,854, Fodor et al, U.S. Pat. Nos. 5,800,992; 5,445,934; and 5,744,305; fluid channel-delivery methods, e.g.

Southern et al, Nucleic Acids Research, 20: 1675-1678 and 1679-1684 (1992); Matson et al, U.S. Pat. No. 5,429,807, and Coassin et al, U.S. Pat. Nos. 5,583,211 and 5,554,501; spotting methods using functionalized oligonucleotides, e.g. Ghosh et al, U.S. Pat. No. 5,663,242; and Bahl et al, U.S. Pat. No. 5,215,882; droplet delivery methods, e.g. Brennan, U.S. Pat. No. 5,474,796; and the like. The above patents disclosing the synthesis of spatially addressable microarrays of oligonucleotides are hereby incorporated by reference.

The number of hybridization sites on planar microarrays may be equivalent in number to the size of the repertoire being employed, since the tag complements on such microarrays are not sampled as they are with microbead arrays. That is, tag complements are synthesized or spotted at predetermined addresses on all the microarrays. Identical copies of planar microarrays may be manufactured so that the same tag complement will be located at the same address for all of the microarrays. This permits multiple hybridization reactions to be carried out simultaneously so that sequence information may be obtained from each size class of fragment of an entire size ladder in the time it takes to carry out a single hybridization reaction, as illustrated in FIG. 1b. Preferably, microarrays used with the invention contain from 5,000 to 500,000 hybridization sites; and more preferably, they contain from 10,000 to 250,000 hybridization sites. In accordance with the invention, the number of microarrays used is usually equal or less than the number of size classes generated in the size ladders. Preferably, this number is in the range of from 12 to 100; more preferably, it is in the range of from 12 to 60; and most preferably, it is in the range of from 16 to 36.

Guidance for selecting conditions and materials for applying labeled oligonucleotide probes to microarrays may be found in the literature, e.g. Wetmur, Crit. Rev. Biochem. Mol. Biol., 26: 227-259 (1991); DeRisi et al, Science, 278: 680-686 (1997); Chee et al, Science, 274: 610-614 (1996); Duggan et al, Nature Genetics, 21: 10-14 (1999); Schena, Editor, Microarrays: A Practical Approach (IRL Press, Washington, 2000); and like references.

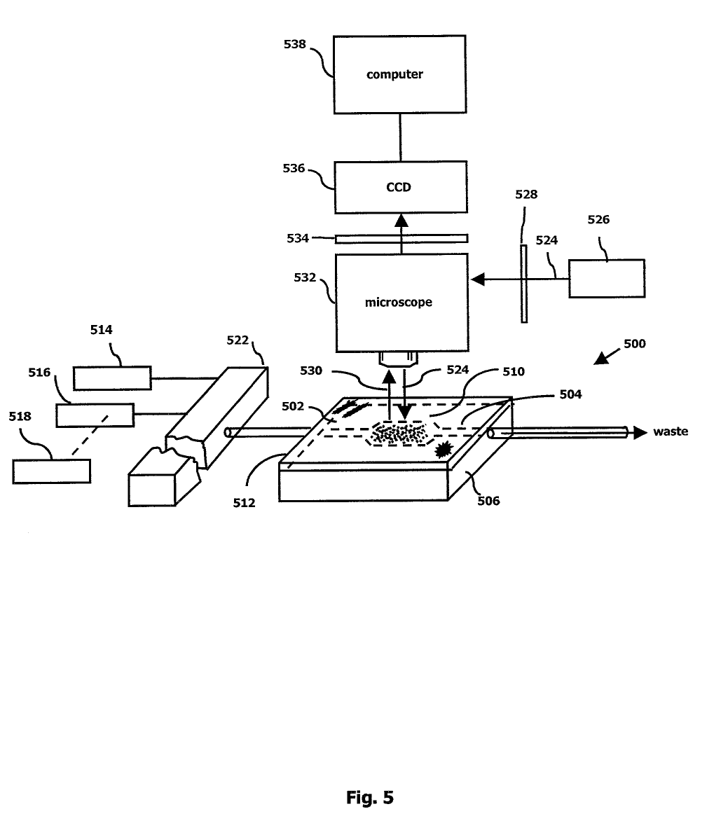

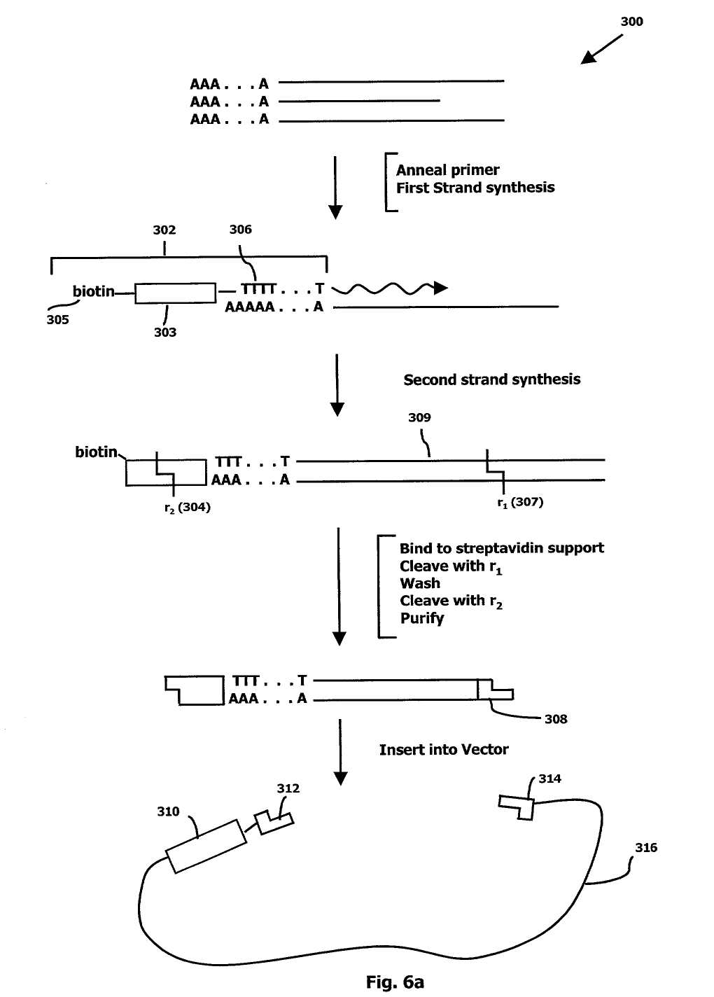

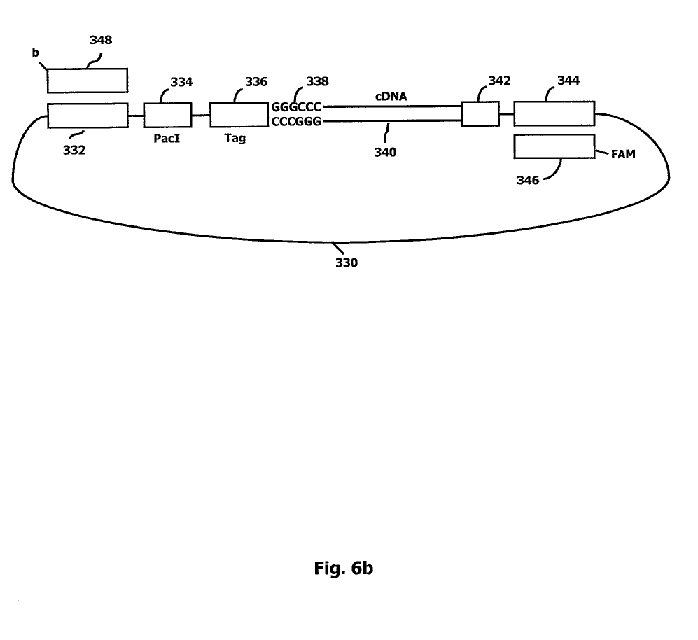

Instruments for measuring optical signals, especially fluorescent signals, from labeled tags hybridized to targets on a microarray are described in the following references which are incorporated by reference: Stern et al, PCT publication WO 95/22058; Resnick et al, U.S. Pat. No. 4,125,828; Karnaukhov et al, U.S. Pat. No. ,354,114; Trulson et al, U.S. Pat. No. 5,578,832; Pallas et al, PCT publication WO 98/53300; and the like. An exemplary instrument for carrying out hybridization reactions on microbead arrays is shown in FIG. 5, and is disclosed in detail in Pallas et al (cited above) and Brenner et al, Nature Biotechnology, 18: 630-634 (2000).

### Schemes for Generating Size Ladders

An important feature of the invention is the generation of a size ladder of polynucleotide fragments for each tag-polynucleotide conjugate of a sample. Preferably, this step can be accomplished in at least two ways: First, the sample can be separated into a plurality of aliquots after which each aliquot undergoes different processing steps to produce a different size class of polynucleotide fragment. Thus, each aliquot will have only a single size class without physical separation. Second, the entire sample can be processed to produce a mixture of size classes of polynucleotide fragments after which the mixture is subjected to a physical separation process to isolate the different size classes.

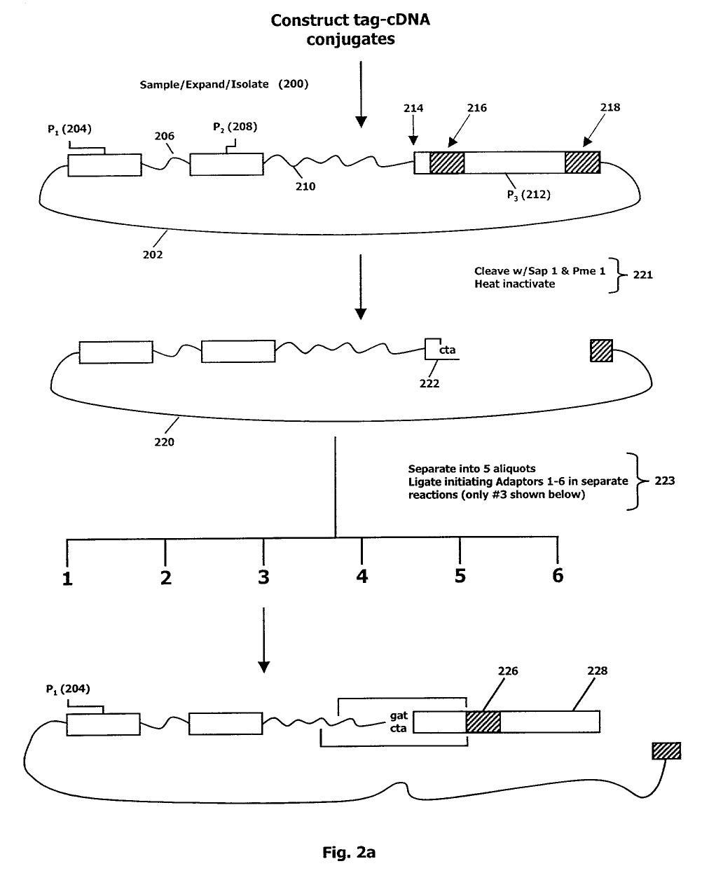

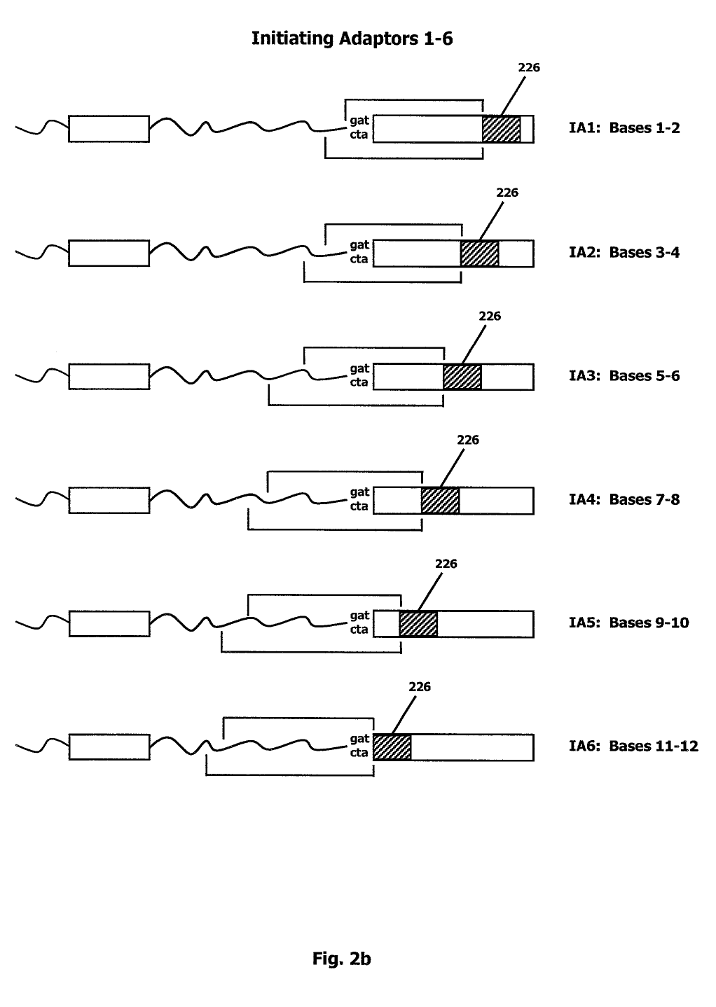

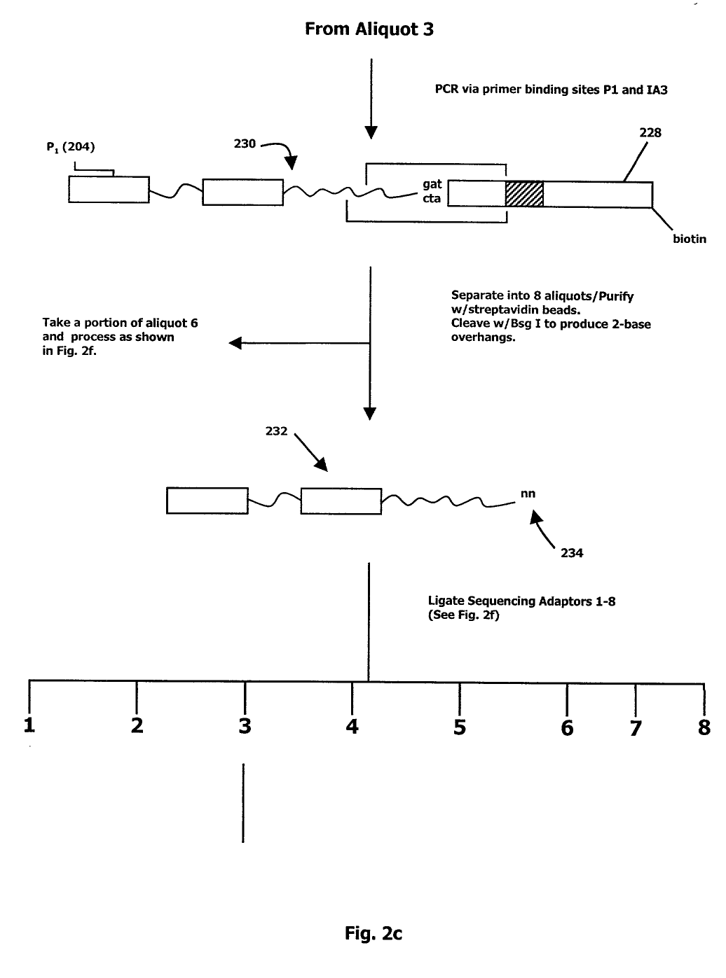

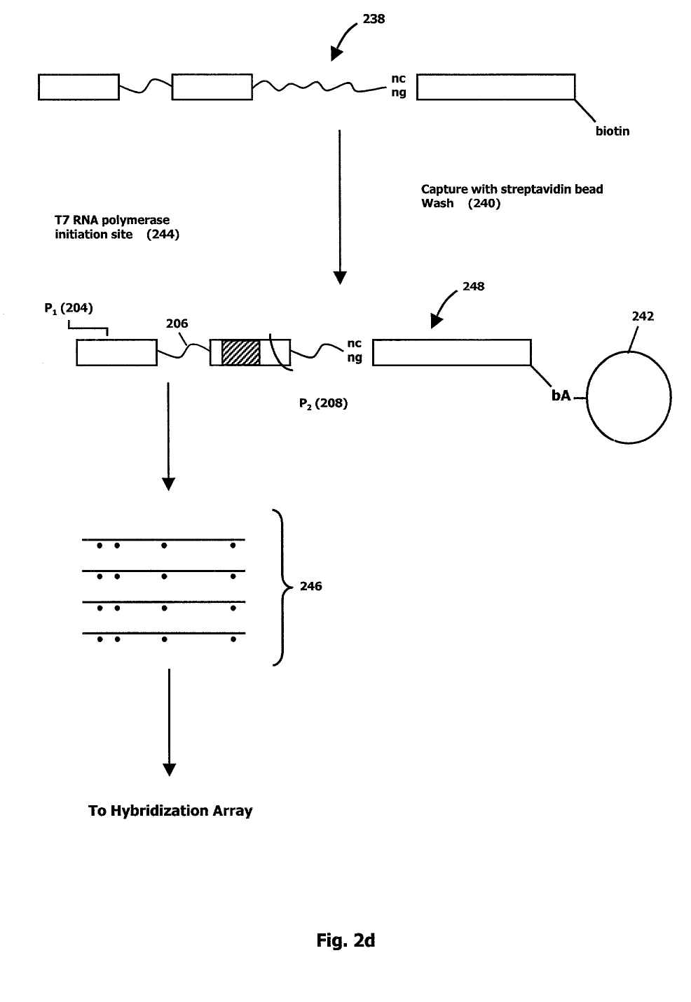

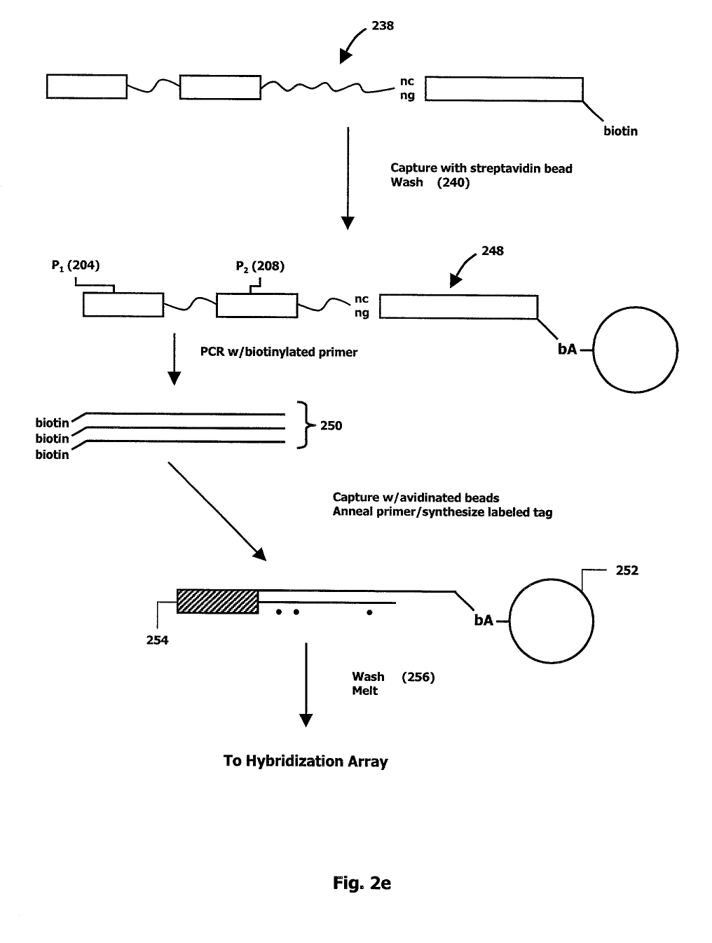

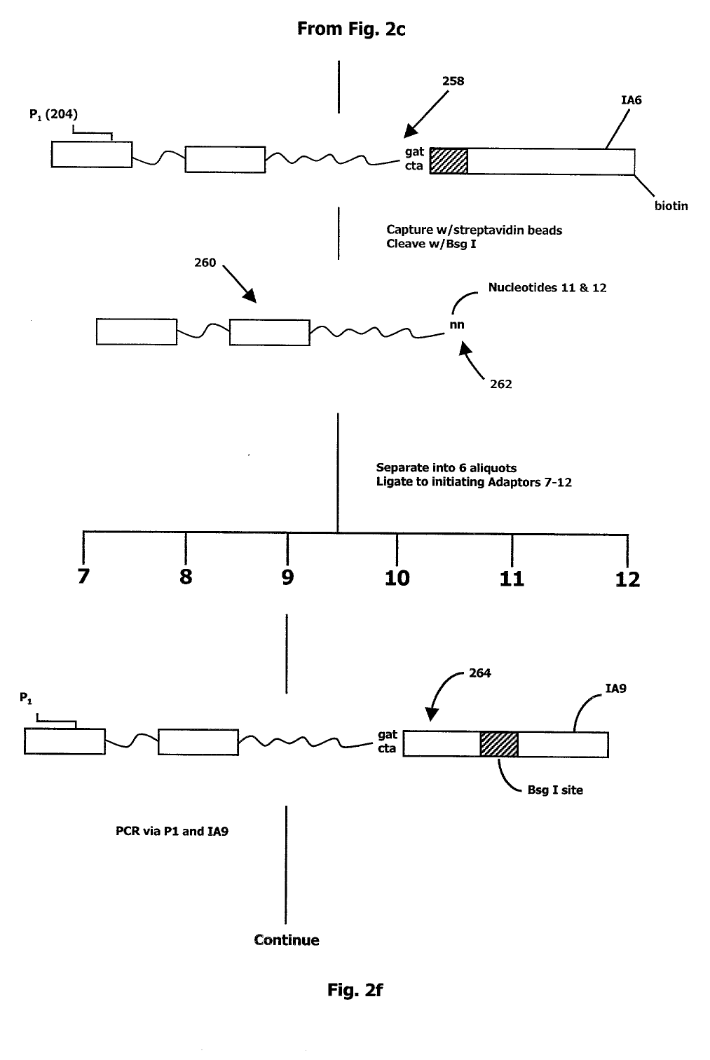

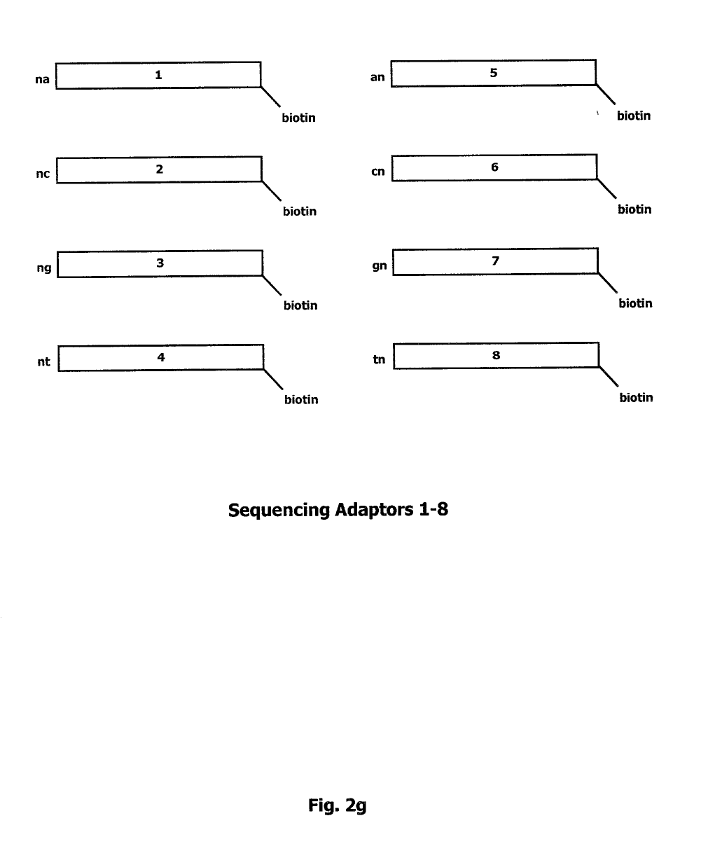

In one aspect of the invention, size ladders are generated by successive cleavages of tag-polynucleotide conjugates with a type IIs restriction endonuclease, followed by the identification of nucleotides in the resulting polynucleotide fragments by the ligation of sequencing adaptors. An example of such an embodiment is illustrated in FIGS. 2a-2f. In FIG. 2a, tag-polynucleotide conjugates are sampled, expanded, and isolated (200) as disclosed in Brenner et al, U.S. Pat. No. 5,846,719, and Brenner et al, Proc. Natl. Acad. Sci., 97: 1665-1670 (2000), to give a mixture of vectors (202) containing the tag-polynucleotide conjugates. As in these references, polynucleotides or cDNAs (210) are directionally cloned into a vector carrying the tags so that one end of the polynucleotides or cDNA has a Dpn II compatible cleavage. This is merely one of many ways to design such a vector, which is well known by one of ordinary skill in the art, and the use of Dpn II is not intended to be limiting. In series, vectors (202) contain primer binding site p₁ (204), tag (206), primer binding site p₂ (208), polynucleotide or cDNA (210), Dpn II site (214), and primer binding site p₃ (212). Primer binding site p₃ further includes type IIs restriction site Sap I (216) positioned to cleave within the Dpn II site and 8-mer restriction site Pme I (218). Again, the use of Sap I and Pme I is a design choice and one skilled in the art would know to use alternative enzymes under different circumstance or conditions. Vectors (202) are cleaved (221) with Sap I and Pme I using conventional protocols to give open vector (220) having 3-mer protruding strand (222), which is a portion of Dpn II site (214). Open vector (220) is separated into six aliquots (223) and in six separate reactions, initiating adaptors 1-6 (shown in FIG. 2b) are ligated onto protruding strand (222) of open vector (220). Initiating adaptors IA1 through IA6 are identical except for the position of type IIs restriction site (226), which as shown, preferably has a reach of (16/14) and therefore leaves a two-nucleotide overhang after cleavage. Exemplary type IIs restriction endonucleases having this property include Bsg I. Preferably, such type IIs site is positioned so that cleavage in initiating adaptor IA1 occurs immediately adjacent to Dpn II site (222) to reveal nucleotides 1 and 2 of the signature sequence. Similarly, the site is positioned in initiating adaptor IA2 so that cleavage reveals the next two nucleotides, that is, nucleotides 3 and 4 of the signature, and so on, for initiating adaptors IA3 through IA6. Returning to FIG. 2a, as shown for reaction number 3, tag (206) and polynucleotide (210) are amplified by PCR using primers that anneal to primer binding site p₁ (204) and initiating adaptor AI3 (228) to give amplicon (230) of FIG. 2c. Amplicon (230) is separated into 8 aliquots, and similar operations are carried out for the aliquots 1, 2, 4, 5, and 6 of FIG. 2a. After affinity purification with streptavidin beads using conventional protocols, the polynucleotide fragments are cleaved with type IIs restriction endonuclease (226) to release polynucleotide fragments (232) with 2-mer protruding strand (234). Sequencing adaptors 1 through 8 (illustrated in FIG. 2g) are ligated to a different one of each of the fragments of aliquots 1 through 8. As indicated by the “n” in the protruding strands shown in FIG. 2g, sequencing adaptors 1 through 8 are each equimolar mixtures of four adaptors. If there is a perfect match between the two-nucleotide overhang of the sequencing adaptor and the fragment, then ligation will be successful; otherwise, no ligation will occur and the fragment will be absent from subsequent steps. Preferably, each of the sequencing adaptor mixtures further includes equimolar concentrations of non-biotinylated adaptors having two-nucleotide overhangs of the form: “n(not a)-” or using the single letter codes for nucleotides, “nb-,” “n(not c)” or “nd”, “n(not g)” or “nh”, and so on. The presence of such adaptors prevents the spurious ligation of the biotinylated adaptors to incorrect overhangs.

Successful ligation leads to ligation product (238) of FIG. 2d, which is purified with streptavidin beads (240). Fragments (248) that are successfully captured by streptavidin beads (242) will have a “c” in position 5 of its signature and an “n” in position 6. In this embodiment, the “n” of position 6 will be identified in the ligation reaction between fragment (232) and sequencing adaptor 7.

Returning to FIG. 2d, amplification by T7 RNA polymerase is one way in which labeled tags may be generated from the captured fragments. Using conventional protocol, placement of T7 RNA polymerase recognition site (244) in primer binding site p₂ (208) permits tag (206) to be amplified and labeled. After amplification in the presence of at least one label ribonucleoside triphosphate, labeled tags (246) may be applied to a hybridization array. Alternatively, tags may be labeled using PCR as shown in FIG. 2e. Starting with amplicon (238), successfully ligated sequencing adaptors are used to capture fragments (248) on streptavidin beads as described above. In this case, primers (one of which is biotinylated) specific for primer binding sites p₁ (204) and p₂ (208) are used to amplify tag (206). Amplified tags (250) are then captured with streptavidin beads (252) and washed. Primer (254) is then annealed to primer binding site p₁ (204) and extended with a DNA polymerase in the presence of a labeled deoxynucleoside triphosphate. After washing, the labeled extension products are melted (256) from the streptavidin beads and applied to the hybridization array.

Returning briefly to FIG. 2c, the operations described above provide for the identification of nucleotides at positions 1-12 of the signature sequences. Further nucleotide can be identified by taking a portion of the fragments of aliquot 6 (those fragments having had the greatest number of nucleotides removed by the cleavage step of FIG. 2c) and religating the six initiating adaptors. In other words, fragment (258) is captured with streptavidin beads and cleaved with a (16/14) type IIs restriction endonuclease, such as Bsg I, to release fragments (260). As shown, the protruding nucleotides are in positions 11 and 12 of the signature sequence. Fragment (260) is separated into 6 aliquots and initiating adaptors AI7 through AI12 (which are identical to AI1 through AI6, respectively) are separately ligated to protruding strand (262) to produce fragments (264), of which only that from aliquot 9 is shown. The fragments are then processed as described above.

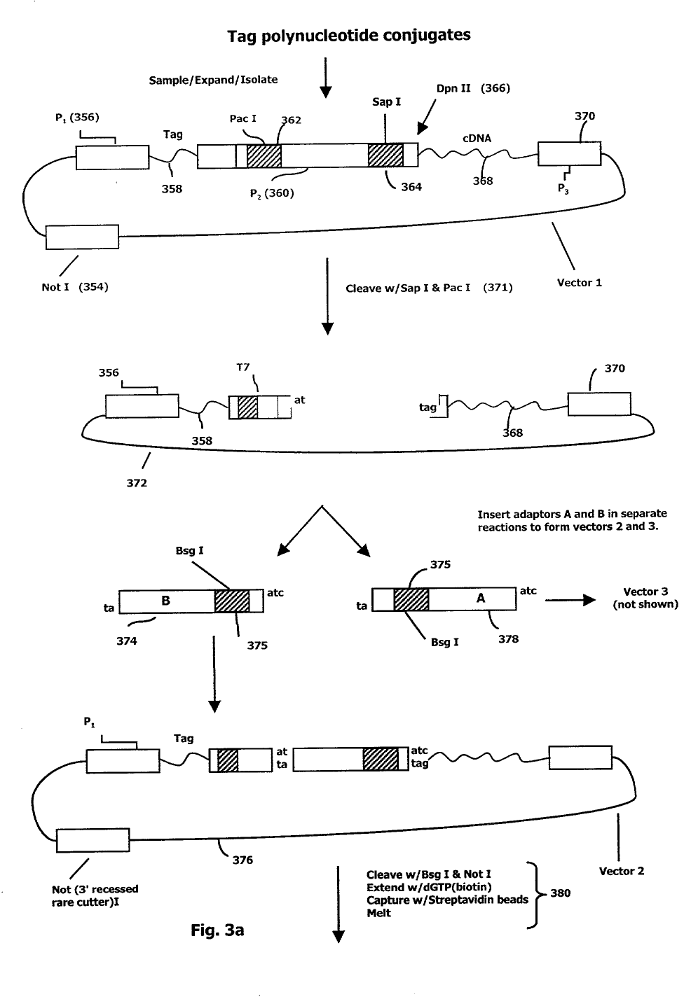

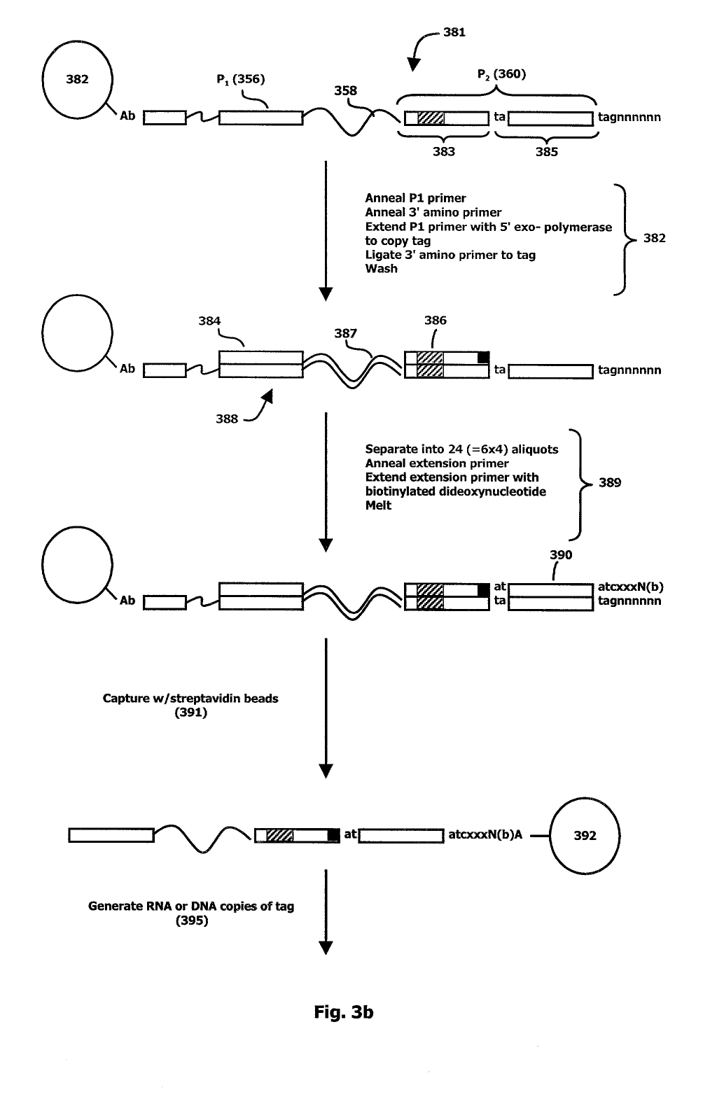

An embodiment for generating size ladders using both cleavage with a type IIs restriction endonuclease and polymerase extension is illustrated in FIGS. 3a and 3b. As above, a sample of tag-polynucleotide conjugates is expanded and isolated (350) using conventional molecular biology techniques to give vector 1 (352) which comprises the following elements in series: a restriction site (354) for a infrequent or rare cutting endonuclease that leaves a 3′ recessed strand after cleavage, such as Not I, or the like; primer binding site p₁ (356); tag (358); primer binding site p₂ (360) containing rare cutting restriction site (362), such as a Pme I site, and type IIs restriction site (364), such as an Sap I site; a Dpn II or like site (366); polynucleotide (368), such as a cDNA; and primer binding site p₃ (370). Again, the Sap I site is positioned so that Sap I cleaves within the Dpn II site to leave a three-nucleotide protruding strand. Vector 1 is divided into two portions in about a 2:1 molar ratio. The smaller portion is set aside for later processing, and additional vectors 2 and 3 are produced from the larger portion. Vectors 2 and 3 of the larger portion are cleaved (371) with Sap I and Pac I to produce opened vector (372), which is then divided into two aliquots. Adaptor B (374) is inserted into opened vector (372) of one aliquot to produce closed circle vector 2 (376), and adaptor A (378) is inserted into open vector (372) of the other aliquot to produce another closed vector 3 (not shown). In order to produce sufficient material for the subsequent processing steps, vectors 2 and 3 may be used to transfect a host, expanded in culture, and re-isolated using conventional protocols. Adaptors A and B are identical except for the position of (16/14) type IIs restriction site (375), which may be Bsg I or like enzyme.

In separate sets of reactions, each of the three vectors are processed as follows (380): cleavage with type IIs restriction endonuclease recognizing (375) and restriction endonuclease recognizing (354) to produce an opened vector having a 3′-protruding strand on an end interior to polynucleotide (368) and a 3′-recessed strand at the opposite end; extend the 3′-recessed strand with a DNA polymerase in the presence of a biotinylated deoxynucleoside triphosphate (which for Not I as (354) is biotinylated guanidine triphosphate); capturing the extended strands with streptavidin beads; and melting off the non-biotinylated strand to produce captured strands (381) shown in FIG. 3b. Preferably, after capture of the biotinylation molecule, streptavidin of the bead is saturated with biotin to preclude any further capturing of biotinylated DNA, the desirability of which will be clear below. The nucleotide sequence “tagmmnnn” distal to the streptavidin bead (382) consists of a portion of Dpn II site (366) and six nucleotides of polynucleotide (368). To the captured DNA strands (381) the following steps (382) are applied: anneal primer p₁ (384) to primer binding site p₁ (356), anneal 3′-amino primer (386) to region 1 (383) of primer binding site p₂ (360); extend primer p₁ with a DNA polymerase lacking 5′ exonuclease activity to copy tag (358); ligate 3′-amino primer (386) to copied strand of tag (387); and wash, to give structure (388). 3′-amino primer is a primer whose 3′ nucleotide has an amino group substituted for the hydroxyl group at the 3′ carbon position, as taught by Fung et al, U.S. Pat. No. 5,593,826. Such primers cannot be extended by conventional DNA polymerases; however, they can be ligated using conventional ligases to adjacent oligonucleotides or other strands annealed to the same template. Thus, primer p₁ (384) can be extended to copy tag (358), but 3′-amino primer (386) will not be extended in the reaction, thereby leaving region 2 (385) of primer binding site p₂ (360) single stranded. After the extension reaction, 3′-amino primer can then be ligated to the copied tag (387). Structure (388) is separated into 24 (=6×4) aliquots (389), one aliquot for each of the four possible nucleotides and for each of the six possible positions from which the extension will take place. In each aliquot, extension primers (390) are annealed to the single stranded portion of the attached DNA under stringent conditions so that only perfectly matched duplexes are formed, after which the extension primers are extended a single nucleotide using a DNA polymerase in the presence of mixture of the four dideoxynucleoside triphosphates one of which is biotinylated. Extension primers have the following form:
atnnnn . . . nnnatc(x)ₛ
   where s is an integer between 0 and 5, inclusive, and x is a so-called “universal” base that is able to form a basepair with more than one of the four natural nucleotides, and preferably any of the four natural nucleotides. Such universal nucleotides serve as “spacers” that allows extension products to be generated at different positions along a polynucleotide. Many such universal nucleotides can be employed, as disclosed in U.S. Pat. No. 5,002,867. Preferably, 3-nitropyrrole or 5-nitroindole substituted nucleotides are employed, which are described in Nichols et al, Nature, 369: 492-493 (1994); Loakes et al, Nucleic Acids Research, 22: 4039-4043 (1994); Bergstrom et al, J. Am. Chem. Soc., 117: 1201-1209 (1995); and which are available from Glen Reseach. After extension, the strands not covalently linked to streptavidin beads (382) are melted, separated from beads (382), and captured with streptavidin beads (392). As above, after the initial template is captured on streptavidin beads (380), remaining site are saturated with biotin, so that the extended strand (390) is not captured by bead (382). The captured strands are then used to generate labeled tags (395) as described above, after which they are applied to one or more hybridization arrays. Preferably, kit for practicing this embodiment include tag-containing vectors for generating tag-polynucleotide conjugates with appropriate primer binding or polymerase binding sites, e.g. as illustrated in FIGS. 3a and 3b, 3′-amino primers, and extension primers. More preferably, such kits further include streptavidin bead, 5′-exo⁻ DNA polymerase, means for generating labeled oligonucleotide tags comprising either primers and DNA polymerase for PCR amplification (as illustrated in FIG. 2e) or an RNA polymerase and labeled ribonucleoside triphosphates, and a plurality of microarrays.

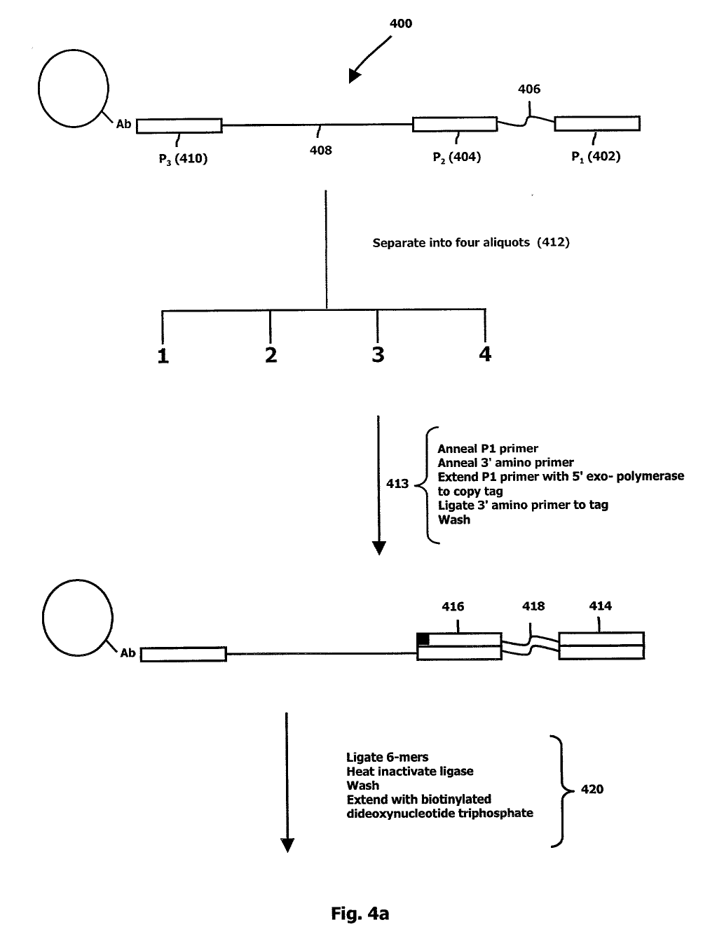

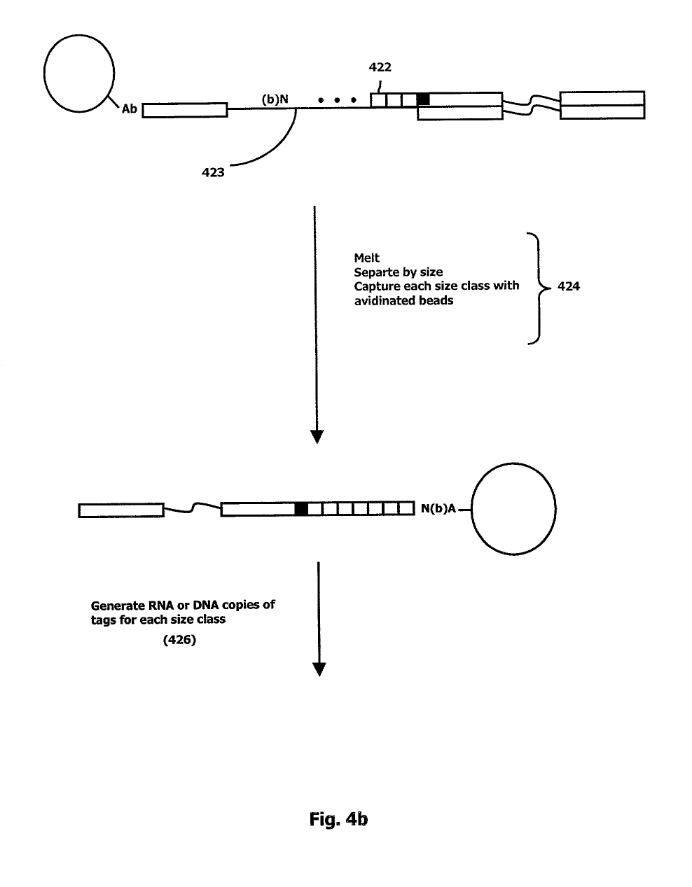

In a further embodiment of the invention, size ladders are generated by extending a primer by ligating oligonucleotides (“extension oligonucleotides”) of the same known length, as illustrated in FIGS. 4a and 4b. Such extension oligonucleotides have sequences that permit the formation of perfectly matched duplexes of any sequence the length of the extension oligonucleotide. In one embodiment, this condition is accomplished by providing mixtures of extension oligonucleotides, wherein extension oligonucleotides of every sequence is represented in the mixture. Thus, if extension oligonucleotides are 4-mers, then the mixture will have 4⁴=256 components. Extending primers by ligating oligonucleotides that anneal to a template is well-known in the art and guidance for selecting specific conditions is provided in the following references, which are incorporated by reference: Blocker, U.S. Pat. No. 5,114,839; Brennan et al, U.S. Pat. No. 5,403,708; Macevicz, U.S. Pat. No. 5,750,341; Kaczorowski and Szybalski, Gene, 179: 189-193 (1996); Gene, 176: 195-198 (1996); and Gene 223: 83-91 (1998). Oligonucleotides of a variety of different lengths may be used, provided that they have the same length in a particular embodiment. Oligonucleotide having lengths between 2 and 10 nucleotides, inclusive, can be used. Preferably, the length of oligonucleotides is in the range of from 5 to 8 nucleotides, inclusive; and most preferably, the length of the oligonucleotide is six nucleotides. As mentioned above, oligonucleotides used in the extension reaction must included sequences that are complementary to every possible sequence that can occur on the polynucleotide template (or tag-polynucleotide template in some embodiments). The oligonucleotides may consist of the four natural nucleotides, or they may contain, or consist entirely of, universal nucleotides. Preferably, the oligonucleotides contain a predetermined number of universal nucleotides between 1 and 3. Thus, for 6-mer oligonucleotides having all four nucleotides there will be 4⁶ (=4096) 6-mers in the extension reaction. In a complexity reducing analog, such as inosine is used which can basepair with either C or A, then there will be 3⁶ (=729) 6-mers in the extension reaction. For 6-mers comprising two truly universal nucleotides, there will be 4⁴ (=256) 6-mers in the extension reaction. In accordance with this embodiment, after the ligation extension reactions are halted, a set of polynucleotide fragments are created each differing in length from one another by integral multiples of the length of the extension oligonucleotide. Preferably, each such fragment is then extended one nucleotide further using a DNA polymerase in a conventional reaction. Preferably, the incorporated nucleotide contains a label or other moiety, such as biotin, from which the identity of the nucleotide can be determined, as exemplified below.

Going now to FIG. 4a, structure (400) containing polynucleotide (408) and tag (406) is produced by amplifying a segment of a vector similar to vector 1 of FIG. 3a using a primer specific for primer binding site p₁ (402) and a primer specific for primer binding site p₃ (410). The region consisting of primer binding site p₁ (402), tag (406), and primer binding site p₂ (404) may be employed similarly as the “binding region” of Macevicz, U.S. Pat. No. 5,750,341.

When conventional ligases are employed in the invention, the 5′ end of the oligonucleotides are phosphorylated. A 5′ monophosphate can be attached to an oligonucleotide either chemically or enzymatically with a kinase, e.g. Sambrook et al, Molecular Cloning: A Laboratory Manual, 2nd Edition (Cold Spring Harbor Laboratory, New York, 1989). Chemical phosphorylation is described by Horn and Urdea, Tetrahedron Lett., 27: 4705 (1986), and reagents for carrying out the disclosed protocols are commercially available, e.g. 5′ Phosphate-ON™ from Clontech Laboratories (Palo Alto, Calif.). Preferably, when required, oligonucleotide probes are chemically phosphorylated.

Generally, when an oligonucleotide anneals to a template in juxtaposition to an end of an extended duplex, the duplex and oligonucleotide are ligated, i.e. are caused to be covalently linked to one another. Ligation can be accomplished either enzymatically or chemically. Chemical ligation methods are well known in the art, e.g. Ferris et al, Nucleosides & Nucleotides, 8: 407-414 (1989); Shabarova et al, Nucleic Acids Research, 19: 4247-4251 (1991); and the like. Preferably, enzymatic ligation is carried out using a ligase in a standard protocol. Many ligases are known and are suitable for use in the invention, e.g. Lehman, Science, 186: 790-797 (1974); Engler et al, DNA Ligases, pages 3-30 in Boyer, editor, The Enzymes, Vol. 15B (Academic Press, New York, 1982); and the like. Preferred ligases include T4 DNA ligase, T7 DNA ligase, E. coli DNA ligase, Taq ligase, Pfu ligase, and Tth ligase. Protocols for their use are well known, e.g. Sambrook et al (cited above); Barany, PCR Methods and Applications, 1: 5-16 (1991); Marsh et al, Strategies, 5: 73-76 (1992); and the like. Generally, ligases require that a 5′ phosphate group be present for ligation to the 3′ hydroxyl of an abutting strand.

Returning to FIG. 4a, structure (400) is separated into four aliquots (412), after which each is treated (413) identically as follows, except for the type of biotinylated dideoxynucleoside triphosphate added. As with the embodiment of FIGS. 3a and 3b, primer p₁ (414) is annealed to primer binding site p₁, 3′-amino primer (416) is annealed to primer binding site p₂ (404), primer p₁ (414) is extended with a DNA polymerase lacking 5′ exonuclease activity, after which 3′-amino primer (416) is ligated to extended strand (418). After washing, 3′-amino primer (416) is extended (420) in each of the four reactions by oligonucleotide ligation under conditions disclosed by Kaczorowki and Szybalski (cited above). Preferably, the reaction is timed so that the extension products are in the range of from about 50 to 120 nucleotides. Clearly, the conditions selected to give such results will take into account the lengths of the various primers and the length of the tag. Reaction time may be controlled by using a ligase that is readily inactivated by heating. As above, after capture of the construct (400), remaining streptavidin sites on the bead are saturated with biotin.

Preferably, after the ligation reaction has been stopped, the extension products are extended further (420) by a single biotinylated dideoxynucleotide to give a final biotinylated extension product (422). Extension product (422) is melted off of the covalently attached strand (423) and separated by size, as described below. Each of the separated size classes is then captured with streptavidinated beads, as described for the embodiment of FIGS. 3a and 3b, after which labeled tags are generated (426), also as described above.

As above, preferably, kits for practicing this embodiment include tag-containing vectors for generating tag-polynucleotide conjugates with appropriate primer binding or polymerase binding sites, e.g. as illustrated in FIGS. 4a and 4b, 3′-amino primers, and oligonucleotide mixtures for generating extension products. More preferably, such kits further include streptavidin beads, 5′-exo⁻ DNA polymerase, means for generating labeled oligonucleotide tags comprising either primers and DNA polymerase for PCR amplification (as illustrated in FIG. 2e) or an RNA polymerase and labeled ribonucleoside triphosphates, and a plurality of microarrays.

### Separation of Size Ladders by Denaturing HPLC

The following describes a procedure for size-based and sequence-independent separation and purification of groups of oligonucleotides from PCR amplified library mixtures, containing extension products from approximately 50 to 100 bases in length. Each separated group of oligonucleotides differs by size from other groups by multiples of six bases and each group comprises a library of identical base-length single-stranded oligonucleotides, which may vary from each other in sequence through the entire length of the DNA. This procedure affords preparative resolution by base-size of the oligonucleotides in the mixture, with size-based purities of 80% or greater, for subsequent sequencing.

Preferably, this purification is performed by integrated high performance liquid chromatography (HPLC) with a detector-coupled fraction collector and with column and mobile phase gradients optimized for the separation of DNA components into microwell plates. As necessary, separation may employ either diethyl amino ethane (DEAE) anion exchange chromatography, or ion-pairing Reverse-Phase chromatography, or a combination of both to effect the purification. The separation is performed on samples containing as little as 1 nanogram (ng) of each base-size group of oligonucleotides, and containing as much as 1 μg total oligonucleotides, and on samples containing as many as 50 sizes of oligonucleotides to be separated.

The procedure utilizes the following equipment and reagents:
1. High Pressure Liquid Chromatograph—HP1100 (Agilent Technologies) or equivalent, with a minimal configuration consisting of a binary pump, UV detector, Column Heater, and Injection System
2. 96-well based Fraction Collection System, with automated peak detection based control of fraction collection. Manual fraction collection may be substituted.
3. DEAE Ion Exchange Chromatography:
   Column—Dionex DNA-PAC (or equivalent)
   HPLC Solvents—
      A) Distilled, deionized water (dH2O)
      B) Sodium perchlorate (0.375M in dH2O)
      C) Sodium chloride (2M in dH2O)
   Typical Conditions—Solvent Flow at 1.0 mL/min., Detector at 260 nm, Column oven at 50° C. Initial solvent conditions are 0% Solvent B and 100% of Solvent A. Upon injection of sample, solvent programmed linearly to 80% B in 60 minutes. Solvent C may be used to optimize separations. Conditions are optimized to provide maximal separation by oligonucleotide size, while minimizing sequence-based separation.
4. Ion Pairing Reverse Phase Chromatography:
   Column—Zorbax Eclipse-DNA column (Agilent Technologies), or equivalent
   Ion Paring Reagent—Tetraalkyl ammonium bromide, where the alkyl group is typically tetra butyl, however tetra hexyl-, or tetra octyl- may be substituted to obtain optimal separation for a particular library.
   HPLC Solvents—
      A) Distilled, deionized water (dH2O) with typically 0.1M ion pairing agent (adjusted for optimal separation for a particular library)
      B) Acetonitrile (ACN) with typically 0.1M ion pairing agent (adjusted as above)
   Typical Conditions—Solvent Flow at 1.0 mL/min., Detector at 260 nm, Column oven at 50° C. Initial solvent conditions are 20% Solvent B and 80% of Solvent A. Upon injection of sample, solvent programmed linearly to 80% B in 60 minutes. Conditions are optimized to provide maximal separation by oligonucleotide size, while minimizing sequence-based separation.
      Procedure:

Samples are concentrated to approximately 0.10 to 1.00 μg total DNA in 20 μL. The HPLC is typically setup using the ion-pairing reverse phase chromatographic conditions above. The 20 μL sample is injected upon the HPLC and the detector output (at 260 nm) is tracked either manually or via computer to direct samples eluting from the column either to waste (before the samples start to elute) or to the microplate fraction collector. At start of elution of DNA peaks, samples are collected, at minimum, one fraction per peak as observed on the HPLC detector output. After elution of constituent DNA peaks, the HPLC column elute is diverted to waste, and the column is washed with 80% of Solvent B.

Alternately, as necessary, a similar procedure is employed with DEAE anion exchange HPLC to pre-separate DNA by size, before transfer of individual eluting peaks to ion pairing reverse phase HPLC for final separation and collection as described above. The procedure may be performed manually or by computer controlled column switching to automate the 2-dimensional size-based purification of DNA libraries.

After collection, DNA size-separated fractions, are purified and concentrated for use in sequencing.

### Instrumentation for Hybridizing Labeled Tags to an Array of Microbeads

Several instruments are available for implementing the method of the invention. In particular, instruments used for hybridizing fluorescent probes to microarrays may be used with the present invention, such as disclosed in U.S. Pat. No. 5,992,591, or like instrument.

When an array of microbeads is used as solid phase supports, apparatus as described in Interntional application PCT/US98/11224 or Brenner et al, Nature Biotechnology, 18: 630-634 (2000), may be used. A flow chamber (500), diagrammatically represented in FIG. 5, is prepared by etching a cavity having a fluid inlet (502) and outlet (504) in a glass plate (506) using standard micromachining techniques, e.g. Ekstrom et al, International patent application PCT/SE91/00327; Brown, U.S. Pat. No. 4,911,782; Harrison et al, Anal. Chem. 64: 1926-1932 (1992); and the like. The dimension of flow chamber (500) are such that loaded microbeads (508), e.g. GMA beads, may be disposed in cavity (510) in a closely packed planar monolayer of 500 thousand to 1 million beads. Cavity (510) is made into a closed chamber with inlet and outlet by anodic bonding of a glass cover slip (512) onto the etched glass plate (506), e.g. Pomerantz, U.S. Pat. No. 3,397,279. Reagents are metered into the flow chamber from syringe pumps (514 through 520) through valve block (522) controlled by a microprocessor as is commonly used on automated DNA and peptide synthesizers, e.g. Bridgham et al, U.S. Pat. No. 4,668,479; Hood et al, U.S. Pat. No. 4,252,769; Barstow et al, U.S. Pat. No. 5,203,368; Hunkapiller, U.S. Pat. No. 4,703,913; or the like.

Hybridization, identification, and washing are carried out in flow chamber (500) to generate signature sequences. Labeled oligonucleotide tags specifically hybridize to tag complements and are detected by exciting their fluorescent labels with illumination beam (524) from light source (526), which may be a laser, mercury arc lamp, or the like. Illumination beam (524) passes through filter (528) and excites the fluorescent labels on tags specifically hybridized to tag complements in flow chamber (500). Resulting fluorescence (530) is collected by confocal microscope (532), passed through filter (534), and directed to CCD camera (536), which creates an electronic image of the bead array for processing and analysis by workstation (538). Preferably, labeled oligonucleotide tags at 25 nM concentration are passed through the flow chamber at a flow rate of 1-2 μL per minute for 10 minutes at 20° C., after which the fluorescent labels carried by the tag complements are illuminated and fluorescence is collected. The tags are melted from the tag complements by passing NEB #2 restriction buffer with 3 mM MgCl₂ through the flow chamber at a flow rate of 1-2 μL per minute at 55° C. for 10 minutes.

### Appendix 1

Fortran Source Code for Calculating the Melting Temperature Distribution of Oligonucleotide Tags Constructed from Four 6-mer Words Selected from Table I

Program sixmer

c
c

| | |
|---|---|
| c | 6mer concatenates four 6-mer words spaced with “a” spacers |
| c | between words. 6mer then calculates the Tm for every |
| c | possible 27-mer and gives the freg. Tm v. Tm for the set. |
| c | |
| c | Melting temperature for an |
| c | oligonucleotide is calc using a standard algorithm, e.g. |
| c | Wetmur, Critical Rev. in Biochem & Mol. Biol. |
| c | 26: 227-259 (1991); |
| c | Rychlik et al, NAR, 18: 6409-6412 (1990); with |
| c | thermodynamic parameters for base stacking enthalpy |
| c | and entropy from |
| c | Breslauer et al, PNAS, 83: 3746-3750 (1986). |
| c | These algorithms and parameters are based on hybridization |
| c | in 1 M NaCl and the assumption that the probe is in |
| c | significant excess of target sequences. Since the 1 M |
| c | NaCl is far in excess of anticipated experimental conditons, |
| c | the Tm's calculated by this program are primarily of |
| c | value for comparisons. |
| c | |

dimension htable(4,4),stable(4,4),otemp(1000000,3)
integer*2 koligo(50),iwords(80,6)
integer ntm1(30),ntm2(30),ntm3(30)
character*15 worddata
common/one/koligo,htable,stable,nseq,otemp,nwords

c

nseq=27

c

| | |
|---|---|
| c | Read thermodynamic parameters. |
| c | |
| c | |

open(1,file=‘h.dat’ ,form=‘formatted’,status=‘old’)
do 100 i=1,4

| | |
|---|---|
| 100 | read(1,101)(htable(i,j),j=1,4) |
| 101 | format(4(f4.1,1x)) |

close(1)

c
c

open(1,file=‘s.dat’,form=‘formatted’,status=‘old’)
do 150 i=1,4

| | |
|---|---|
| 150 | read(1,151)(stable(i,j),j=1,4) |
| 151 | format(4(f5.4,1x)) |

close(1)

c
c

| | |
|---|---|
| c | Read word sequences |
| c | |
| c | a=1, c=2, g=3, & t=4 |
| c | |
| c | |

| | write(*,*)‘Enter worddata file’ |
|---|---|
| | read(*,1990)worddata |
| 1990 | format(a15) |
| | open(1,file=worddata,form=‘formatted’,status=‘old’) |
| | read(1,1991)nwords |
| 1991 | format(i2) |
| | do 190 i=1,nwords |

| | read(1,191)(iwords(i,k),k=1,6) |
|---|---|
| 191 | format(6i1) |
| 190 | continue |
| | close(1) |

c
c

| c | Print words |
|---|---|
| c | |
| c | |
| | write(*,*) |
| 1995 | format(1x,‘nwords=’,i4) |
| | do 193 jj=1,nwords |
| | write(*,192)(iwords(jj,k),k=1,6) |
| 193 | continue |
| 192 | format(5x,6i1) |
| | write(*,1995)nwords |
| | pause |

c
c

open(7,file=‘6dis.dat’,form=‘formatted’,status=‘replace’)

c

| c | Concatenate words to form tags, then |
|---|---|
| c | calculate Tm's |
| c | |
| c | |
| | tmin=1000. |
| | tmax=0. |
| | ntags=0 |
| | do 1000 i1=1,nwords |

do 1000 i2=1,nwords

do 1000 i3=1,nwords

do 1000 i4=1,nwords

ntags=ntags+1

c
c

koligo(1)=iwords(i1,1)
koligo(2)=iwords(i1,2)
koligo(3)=iwords(i1,3)
koligo(4)=iwords(i1,4)
koligo(5)=iwords(i1,5)
koligo(6)=iwords(i1,6)
koligo(7)=1

c

koligo(8)=iwords(i2,1)
koligo(9)=iwords(i2,2)
koligo(10)=iwords(i2,3)
koligo(11)=iwords(i2,4)
koligo(12)=iwords(i2,5)
koligo(13)=iwords(i2,6)
koligo(14)=1

c

koligo(15)=iwords(i3,1)
koligo(16)=iwords(i3,2)
koligo(17)=iwords(i3,3)
koligo(18)=iwords(i3,4)
koligo(19)=iwords(i3,5)
koligo(20)=iwords(i3,6)
koligo(21)=1

c

koligo(22)=iwords(i4,1)
koligo(23)=iwords(i4,2)
koligo(24)=iwords(i4,3)
koligo(25)=iwords(i4,4)
koligo(26)=iwords(i4,5)
koligo(27)=iwords(i4,6)

c

call temp(tt1,tt2,tt3)

c
c

if(tmin.gt.tt1) tmin=tt1
if(tmin.gt.tt2) tmin=tt2
if(tmin.gt.tt3) tmin=tt3
if(tmax.lt.tt1) tmax=tt1
if(tmax.lt.tt2) tmax=tt2
if(tmax.lt.tt3) tmax=tt3

c
c

| | otemp(ntags,1)=tt1 |
|---|---|
| | otemp(ntags,2)=tt2 |
| | otemp(ntags,3)=tt3 |
| | write(*,204)ntags,(otemp(ntags,ix),ix=1,3) |
| 1000 | continue |
| c | |
| c | |
| 204 | format(i9,2x,3(2x,f10.4)) |
| C | |
| c | |
| c | Calculate the distribution of Tm's |
| c | |
| c | |

dt=(tmax−tmin)/30.
do 4100 k=1,30

ntm1(k)=0
ntm2(k)=0
ntm3(k)=0

at=tmin + dt*float(k−1)
bt=tmin + dt*float(k)

c

do 4200 kg=1,ntags

if(otemp(kg,1).ge.at.and.otemp(kg,1).lt.bt) then

ntm1(k)=ntm1(k) + 1
endif

if(otemp(kg,2).ge.at.and.otemp(kg,2).lt.bt) then

ntm2(k)=ntm2(k) + 1
endif

if(otemp(kg,3).ge.at.and.otemp(kg,3).lt.bt) then

ntm3(k)=ntm3(k) + 1
endif

c
c

| | |
|---|---|
| 4200 | continue |
| 4100 | continue |
| c | |
| c | |

| | write(*,4499)tmin,tmax |
|---|---|
| | write(7,4499)tmin,tmax |
| | do 4498 kj=1,30 |
| | write(*,4500)ntm1(kj),ntm2(kj),ntm3(kj) |
| | write(7,4500)ntm1(kj),ntm2(kj),ntm3(kj) |
| 4498 | continue |
| 4500 | format(3(2x,i9)) |
| 4499 | format(1x,‘tmin=’,f6.1,2x,‘tmax=’,f6.1) |
| | close(7) |

c
c

end

c
c
c********************************************************************
c

subroutine temp(tt1,tt2,tt3)

c
c

dimension htable(4,4),stable(4,4),otemp(1000000,3)
integer*2 koligo(50)
common/one/koligo,htable,stable,nseq,otemp,nwords

c
c

dh=0.
ds=0.
r=.00199
conc=.000000001

c
c

| | |
|---|---|
| c | Perfect match: |
| c | |
| c | |

do 2100 iq=1,nseq−1

| | dh=dh + htable(koligo(iq),koligo(iq+1)) |
|---|---|
| | ds=ds + stable(koligo(iq),koligo(iq+1)) |
| 2100 | continue |
| c | |
| c | |

tt1=(dh−5.)/(ds−r*log(conc)) −273.2

c
c

| | |
|---|---|
| c | 3′ Mismatch: |
| c | |
| c | |

dh=0.
ds=0.
do 2200 iq=7,nseq−1

| | dh=dh + htable(koligo(iq),koligo(iq+1)) |
|---|---|
| | ds=ds + stable(koligo(iq),koligo(iq+1)) |
| 2200 | continue |
| c | |
| c | |

tt2=(dh−5.)/(ds−r*log(conc)) −273.2

c
c

| | |
|---|---|
| c | 5′ Mismatch |
| c | |
| c | |

dh=0.
ds=0.
do 2300 iq=1,nseq-7

| | dh=dh + htable(koligo(iq),koligo(iq+1)) |
|---|---|
| | ds=ds + stable(koligo(iq),koligo(iq+1)) |
| 2300 | continue |
| c | |
| c | |

tt3=(dh−5.)/(ds−r*log(conc)) −273.2

c
c

return
end

c********************************************************************

APPENDIX 2
Tm distributions for tags constructed from four 6-mer words
from a minimally cross-hybridizing set of 32 (Table I).
Each 6-mer word differs from every other 6-mer by four bases.
tmin = 57.5 tmax = 81.8

| Perfect Match | 3′-Mismatch | 5′-Mismatch |
|---|---|---|
| 0 | 0 | 864 |
| 0 | 96 | 736 |
| 0 | 2272 | 5024 |
| 0 | 5152 | 15840 |
| 0 | 15776 | 22688 |
| 0 | 39264 | 48576 |
| 0 | 63808 | 94912 |
| 0 | 103328 | 125056 |
| 0 | 160768 | 133536 |
| 0 | 165440 | 191520 |
| 0 | 170688 | 185216 |
| 0 | 177696 | 113312 |
| 19 | 108288 | 66560 |
| 149 | 34656 | 31616 |
| 554 | 1344 | 12352 |
| 2343 | 0 | 768 |
| 6843 | 0 | 0 |
| 16448 | 0 | 0 |
| 36594 | 0 | 0 |
| 66015 | 0 | 0 |
| 102981 | 0 | 0 |
| 144885 | 0 | 0 |
| 169184 | 0 | 0 |
| 168859 | 0 | 0 |
| 148840 | 0 | 0 |
| 101418 | 0 | 0 |
| 54259 | 0 | 0 |
| 21519 | 0 | 0 |
| 6140 | 0 | 0 |
| 1526 | 0 | 0 |

1. A method of simultaneously determining a signature sequence for each polynucleotide in a population of polynucleotides, the method comprising the steps of:
attaching an oligonucleotide tag from a repertoire of tags to each polynucleotide of the population to form tag-polynucleotide conjugates such that substantially every different polynucleotide has a different oligonucleotide tag attached;
generating a size ladder of polynucleotide fragments for each tag-polynucleotide conjugate, each polynucleotide fragment of the same size ladder having an end and the same oligonucleotide tag as every other polynucleotide fragment of the size ladder;
separating the polynucleotide fragments into size classes;
labeling the oligonucleotide tag of each polynucleotide fragment according to the identity of one or more nucleotides at the end of such polynucleotide fragment;
copying the labeled oligonucleotide tags of each polynucleotide fragment of each size class; and
separately hybridizing the labeled oligonucleotide tags of each size class with their respective complements under stringent hybridizaion conditions, the respective complements being attached as populations of substantially identical oligonucleotides in spatially discrete and addressable regions on one or more solid phase supports, and the respective signature sequences being determined by the sequence of labels associated with each spatially discrete and addressable region of the one or more solid phase supports.

2. The method of claim 1 wherein said steps of generating and separating further include forming a plurality of aliquots of tag-polynucleotide conjugates, and shortening by a different amount said polynucleotides of said tag-polynucleotide conjugates in each aliquot such that said polynucleotides in different aliquots are shortened a different amount.

3. The method of claim 2 wherein said step of shortening is carried out enzymatically with a type IIs restriction endonuclease.

4. The method of claim 1 wherein said step of generating further includes forming extension products of known lengths for each tag-polynucleotide.

5. The method of claim 4 wherein said step of generating further includes extending a first primer to copy said tag of each tag-polynucleotide conjugate to form an initializing oligonucleotide and then extending the initializing oligonucleotide by ligating extension oligonucleotides.

6. A method of simultaneously determining a signature sequence of each polynucleotide in a sample of tag-polynucleotide conjugates wherein substantially every different polynucleotide has a different tag, the method comprising the steps of:
generating a size ladder for every tag-polynucleotide conjugate such that each size ladder has a plurality of size classes of polynucleotide fragments;
separating the size classes of polynucleotide fragments;
amplifying and labeling the tag of each polynucleotide fragment according to the identity of one or more nucleotides at an end of each such polynucleotide fragment;
separately hybridizing the labeled tags of each size class with their respective complements under stringent hybridizaion conditions, the respective complements being attached to each of a plurality of microarrays, each microarray of the plurality having the same spatially addressable hybridization sites; and
determining each signature sequence in the sample by a set of signals generated at hybridization sites having the same address on each of the plurality of microarrays.

7. The method of claim 6 wherein said step of generating further includes generating said size ladder for said every tag-polynucleotide conjugate to form a mixture of said size classes.

8. The method of claim 7 wherein said step of separating further includes forming substantially homogeneous populations of each of said size classes of said mixture by physical separation.

9. The method of claim 8 wherein said step of generating further includes extending a first primer to copy said tag of each tag-polynucleotide conjugate to form an initializing oligonucleotide and then extending the initializing oligonucleotide by ligating extension oligonucleotides.

10. The method of claim 9 wherein said step of forming by said physical separation is carried out by preparative gel electrophoresis or HPLC.

11. The method of claim 10 wherein said extension oligonucleotides have a length of from 2 to 10 nucleotides.

12. The method of claim 11 wherein said extension oligonucleotides have a length of from 4 to 6 nucleotides.

13. The method of claim 12 wherein said step of forming by said physical separation is carried out by denaturing HPLC.

14. The method of claim 13 wherein said extension oligonucleotides comprising one or more degeneracy-reducing nucleotide analogs.

15. A kit for simultaneously determining a signature sequence of each polynucleotide in a sample of tag-polynucleotide conjugates wherein substantially every different polynucleotide has a different tag, the kit comprising:
a vector containing a repertoire of oligonucleotide tags for forming tag-polynucleotide conjugates;
extension oligonucleotides for extending an initializing oligonucleotide to generate size ladders of polynucleotide fragments; and
a plurality of microarrays of tag complements.

16. The kit of claim 15 further including labeling means for copying and labeling said oligonucleotide tags.
